(19) United States
(12) Patent Application Publication
(10) Pub. No.: US 2002/0107504 A1
(43) Pub. Date: Aug. 8, 2002

(54) Apparatus for local drug delivery in limb

(76) Inventor: Lucas S. Gordon, Sammamish, WA (US)

Correspondence Address: LAW OFFICES OF RONALD M ANDERSON, 600 108TH AVE, NE, SUITE 507, BELLEVUE, WA, 98004, US

(21) Appl. No.: 09/927,268

(22) Filed: Aug. 9, 2001

Related U.S. Application Data
(63) Continuation-in-part of application No. 09/778,222, filed on Feb. 6, 2001.

(51) Int. Cl. A61M 31/00

(52) U.S. Cl. 604/507; 606/201

(57) ABSTRACT

A method and system for delivering a medicinal agent to a treatment site within a limb of a patient. An infusion catheter is inserted into a blood vessel and advanced to the treatment site. To prevent blood flow through the treatment site from carrying away the medicinal agent, the blood flow in the limb is stopped by applying external pressure with a constriction device, such as a pressure cuff or a tourniquet, and/or with a balloon on the catheter. The medicinal agent is then injected through the catheter. Optionally, the infusion process is repeated in successive cycles that are separated by a rest period in which blood flow in the limb is allowed to resume. A controller automates the process. Preferably a distal constriction device is used to prevent the medicinal agent from flowing out of the treatment area into tissue distal of the treatment area.

### RELATED APPLICATIONS

This application is a continuation-in-part of prior copending U.S. patent application, Ser. No. 09/778,222, filed Feb. 6, 2001, priority in the filing date of which is hereby claimed under 35 U.S.C. § 120.

### FIELD OF THE INVENTION

The present invention is generally directed to a medical method and system to deliver medicinal agents in veins and arteries, and more specifically, is directed to delivery of medicinal agents within specific areas of a patient's limb while reducing concentrations of the medicinal agents within other areas of a patient's body.

### BACKGROUND OF THE INVENTION

Treatment of diseases that affect peripheral areas of a human body is often a difficult task. Advances in cardiology have involved a retrograde (i.e., opposite the direction of blood flow) delivery of medications into areas of the body affected by poor arterial circulation. One such advance is retrograde perfusion, a method of delivering, in the retrograde direction, drugs, solutions, or blood to a tissue area. During cardiopulmonary bypass, retrograde perfusion is sometimes used to deliver cardioplegic solutions into cardiac veins and tissue. In U.S. Pat. No. 4,689,041, entitled, “Retrograde Delivery of Pharmacologic and Diagnostic Agents Via Venous Circulation” (hereinafter “Corday”), Corday et al. describes a method using a catheter having a balloon disposed on its distal end, for retrograde venous injection of various fluids into a blockaded region made inaccessible by an occluded artery. While aiding in the retrograde delivery of fluids into the veins, venules, and capillaries of the heart, Corday does not provide a method for successful retrograde delivery of fluid in other venous systems. Unlike the heart, most other areas of the body have veins that are interconnected to form an outflow path or grid with multiple, parallel, interconnecting vessels. If retrograde perfusion is attempted in these areas using the technique described by Corday et al., the infused fluid merely flows into a parallel vein and away from the capillary vessels, so that retrograde flow of the fluid into the target capillary system does not occur. The capillaries are the optimal blood vessels for drug delivery due to their ultra-thin walls, providing rapid infusion of a drug into the surrounding tissue.

Diabetes, a disease that causes restricted blood flow and arterio-venous shunting in peripheral limbs, leads to infections and ulcers that are slow to heal. Antibiotics applied to the exterior surface of the ulcers have been relatively ineffective due to an inability of the medication to penetrate deeply into tissue surrounding infected areas. An alternative method for treating these infections is a systemic administration of antibiotic agents into the venous system of the infected limb. Unfortunately, concentrations of antibiotics at a level appropriate to treat the infection often cause toxic effects in other parts of the body.

Diabetic foot ulcers have been effectively treated with a regional administration of high concentrations of antibiotics. Cavini-Ferreira et al. report the use of venous infusion of an antibiotic for infected, diabetic foot ulcers (Cavini-Ferreira, P. C., “Retrograde Venous Perfusion in the Diabetic Foot,” Ischemic Diseases and the Microcirculation (1989), K. Messmer. Munchen, pages 92-96) . In part, the method of Cavini-Ferreira uses a technique for circulatory arrest as described by Bier in “Ueber einen neuen Weg Localanasthesie and den Gliedmaassen zu erzeugen,” Archiv klinishe Chirugie 86 (1908), pages 1007-1016. Bier proposed a method of administering local anesthesia to a limb by applying a tourniquet inflated to a pressure above that of the arterial blood flow. The result is complete stasis of the circulatory system in the limb into which an anesthetic agent is then injected, so that the anesthesia is limited to the limb. Cavini-Ferreira et al. use Bier's circulatory arrest technique in conjunction with injection of an antibiotic medication mixed with a large volume of liquid through a needle inserted into a superficial vein on the dorsum of the foot. This treatment is repeated once every 24 hours, for two to eleven days. Each treatment requires a new cannulation of the vein for antibiotic administration. The voluminous injection results in expansion and flooding of the venous system within the foot and leg. The circulatory arrest is maintained for periods of about 20 minutes. When blood flow to an area is reduced or stopped, oxygen deprivation or hypoxia occurs. It is generally believed that ischemia or loss of blood flow to tissue, excluding the heart and brain, may be maintained safely for a period of up to 30 minutes. However, diabetic patients suffer from circulatory abnormalities that include increased arterial-venous shunting in the feet, resulting in lower blood flow to the tissues and hypoxia. This reduction of blood flow is a primary reason that ulcers develop in these locations. It is reasonable to assume that the safe ischemic period for the feet of diabetic patients is much less than 30 minutes and should be minimized.

Blood stasis for long lengths of time may also place the patient at increased risk of thrombosis, although the use of systemic heparinization may lengthen the safe period of arrest. While the extended arrest technique of Cavini-Ferreira et al. is effective in healing infected diabetic foot ulcers, many patients have reported at least moderate pain during the procedure. Cavini-Ferreira et al. do not describe or suggest how this technique could be used for more localized or intermittent drug delivery into the arteries, veins, and capillaries of infected tissue.

U.S. Pat. No. 5,254,087 (McEwan) entitled, “Tourniquet Apparatus For Intravenous Regional Anesthesia” (hereinafter referred to as “McEwan”) describes apparatus designed for administering and maintaining anesthesia (Bier's circulatory arrest) in a portion of a patient's limb, distal to a cuff. The apparatus includes a pressure cuff, transducers for generating a pressure signal representative of the maximum pressure applied to the vein by the cuff, delivery pressure control means responsive to the applied pressure signal for determining a reference pressure and for generating a delivery pressure control signal representative of the reference pressure, and anesthetic delivery means. This system attempts to insure that anesthetic is maintained within the limb during surgery. The anesthetic is delivered into a superficial vein using a cannula; however, McEwan anesthetizes a whole limb, so that blood flow may unnecessarily be interrupted in a region of the limb where the anesthetic is not required. No attempt is made to localize the anesthetic to a particular defined location within the limb. In fact, methods are described for removing as much blood from the limb as possible in order to introduce a maximal amount of anesthetic agent into the entire limb. This approach increases the risk and discomfort involved with denying blood to the limb tissues. Also, McEwan does not describe or suggest administration of any therapeutic or diagnostic agents into the limb, but instead, only describes administration of an anesthetic agent. The long occlusion times required for surgery are acknowledged to be painful to the patient, and methods using dual-bladder cuffs are described to reduce this pain. However, the McEwan patent does not discuss intermittent delivery to prevent or reduce this pain or to reduce the effects of ischemia. This fact is not surprising since the basis of the McEwan patent is to prevent any escape of anesthetic into the general circulation within a patient's body. Generally, this surgical procedure is intended to be performed only once, therefore, McEwan makes no attempt to develop methods or equipment suitable for multiple cannulations of the anesthetic administration site. In any case, it is not desirable to require multiple cannulations because of increased infection risks and discomfort to the patient.

Patents to Calderon, including U.S. Pat. Nos. 4,883,459; 4,867,742; and 4,714,460, disclose various schemes for the retrograde profusion of a tumor using a catheter system that includes a suction lumen and an infusion lumen. Seals are associated with each lumen. The infusion seal includes a balloon that is disposed between an outlet port of the infusion lumen and a port of the suction lumen for use in sealing a patient's vein. Similarly, the suction seal comprises a balloon disposed on the catheter proximal to the port of the suction lumen, for preventing fluid flow through the vein.

In U.S. Pat. No. 4,883,459, Calderon teaches that a carrier medium is injected through the infusion lumen into the vein at a desired flow rate and pressure until a steady-state flow is established. Next, a second, less dense carrier medium is injected through the infusion lumen. The back pressure on the carrier medium is increased when the second carrier medium is at the tumor, forcing the second carrier medium into interstitial spaces in the tumor at an attack site. An active ingredient is then injected behind the carrier fluid into the patient's vein through the infusion lumen and along the established flow path. A back pressure on the carrier fluid and active ingredient is increased when the active ingredient is at the attack site, forcing the active ingredient into the interstitial spaces within the tumor. The active ingredient is then collected through the suction lumen after its profusion through the tumor, preventing the active ingredient, which is a chemotherapy drug of potential toxicity to the remainder of the patient's body, from being circulated throughout the patient's circulatory system. The other two Calderon patents claim various related aspects of this basic concept. This concept helps to localize the treatment. However, the seals may be difficult to establish and maintain accurately, which may allow the injected fluid to leak around the seals. Also, because the blood flow is only stopped in the small area between seals, nearby blood flow may allow the injected fluid to leak away from the tumor through nearby return veins, or beyond the interstitial spaces within the tumor. The suction lumen may also unnecessarily extract blood from nearby vessels rather than just the injected fluid.

U.S. Pat. Nos. 5,069,662 (Bodden); 5,411,479 (Bodden); and 5,817,046 (Glickman) disclose several inventions pertaining to apparatus for isolating fluid flow into and out of the body of a patient, to enable a high concentration of a chemotherapeutic agent to be profused for treating a tumor within the pelvic region of a patient. The two Bodden patents disclose a catheter having spaced-apart balloon sections that can be inflated in a blood vessel to isolate a body organ that contains a tumor. The catheter includes a lumen for withdrawing blood from the organ into which a high concentration of an agent used to treat the cancerous tumor has been injected. Blood is thus extracted from the organ through an isolated section of the vascular system that is coupled to the organ's blood supply and is circulated through a filter to remove the toxic anti-cancer agent before being returned to the patient's body. This process prevents toxic levels of the anti-cancer drug from entering the general circulatory system of the patient. However, because this method employs a high dose of a toxic agent, such as a chemotherapy drug, the method requires that the patient's blood be removed, filtered, and reinfused. The steps involved in carrying out this procedure are relatively complex and not suitable for small, intermittent doses that do not have such adverse effects in general circulation as the highly toxic doses used by Bodden.

A related system is disclosed by Glickman for treating a tumor in the pelvic cavity. In the Glickman invention, bilateral thigh tourniquets are applied to interrupt blood flow into the legs of the patient. Furthermore, balloon catheters are inserted into the patient's body and positioned and inflated to occlude blood flow through the aorta and the vena cava at a point above the pelvic region. Then, additional catheters are inserted to enable blood to be withdrawn from the pelvic cavity and circulated through a filter that removes a chemotherapeutic agent from the blood. Although Glickman mentions the possibility of adapting the apparatus disclosed in his patent to treatment of tumors within other portions of the body, there is no clear explanation of how this object can be accomplished. It also appears that use of Glickman's apparatus for treating tumors in the leg with a balloon catheter and a tourniquet would be of little use in treating a tumor disposed in a foot or other location that could not readily be isolated between a tourniquet and a balloon catheter using his technique.

From the preceding description of various approaches developed in the prior art for isolating and administering treatment to a specific site in a patient's body, it will be evident that there remains a need for a safe and effective method and system that permits the localized and repeated delivery of therapeutic or diagnostic agents into a site and which takes advantage of retrograde perfusion, but avoids the problems occurring due to capillary shunting. In particular, there is a need for a method and system that will enable the localized delivery of a medicinal fluid directly at the site of an infection in capillaries of limbs without causing severe pain to the patient and with little risk of clot formation.

It should be noted that while only a fraction of a patient's total tissue mass may be located distally of a treatment site in a limb, it would still be desirable to isolate a distal portion of tissue from the treatment site to prevent a medical agent delivered to the treatment site from migrating distally to non-target tissue in the limb. Veins are very compliant vessels, capable of expanding to more than 100 percent of their normal volume under internal pressures exceeding 20 millimeters of mercury, and thus, veins distal to the treatment area can accommodate a large volume of medical agent out-flowing from the treatment site unless a distal occlusion is also employed to isolate the treatment site within a limb. It would, therefore, be desirable to provide a method and apparatus adapted to isolate a treatment site in a limb of a patient from non target tissue both proximal and distal to the treatment site, to reduce exposure of non target tissue to a medical agent.

### SUMMARY OF THE INVENTION

The present invention provides a method for delivering a medicinal agent, such as a therapeutic drug or diagnostic agent, to a treatment site within a patient's limb. The method includes the steps of inserting an infusion catheter into the patient's vascular system, either within a vein or an artery, and advancing the catheter to the treatment site. Placement of the catheter in a vein rather than an artery is usually preferred because of easier visualization and access, minimal atherosclerotic plaque buildup, and easier control of bleeding after catheter removal. However, the catheter can also be placed within an artery, if desired. The distal tip of the catheter is advanced as close as possible to capillaries disposed in the treatment site. Thus, in a vein, the distal tip of the catheter is advanced in a retrograde direction relative to normal blood flow, and in an artery, the distal tip is advanced in an antegrade direction relative to the normal direction of blood flow. To prevent loss of the medicinal agent from the treatment site due to blood flow, the blood flow into and out of the treatment site within the limb is stopped by applying external pressure with a constriction device placed on the limb proximal of the treatment site. Preferably, the constriction device is a pressure cuff, tourniquet, or similar device. (Note that as used herein, in regard to a patient's limb, the term “proximal” refers to a location that is closer to the torso of the patient's body than the treatment site, while the term “distal” as applied to a limb refers to a location that is closer to the tip of the limb than the treatment site.)

After the flow of blood has been occluded by the constriction device, a small quantity of the medicinal agent is injected through the catheter. Since the catheter tip is disposed proximate to the treatment site, only a relatively small amount of the medicinal agent need be locally infused into the capillaries to provide the desired levels of the medicinal agent at the treatment site.

Occluding the blood flow into and out of the treatment site of the limb also enables the medicinal agent to perfuse into target tissue of the treatment site more effectively than would a medicinal agent injected in flowing blood, or at a location remote from the treatment site. After a predetermined perfusion period has lapsed, the constriction device is released, allowing normal blood flow through the vascular system to resume, to re-oxygenate the limb. The occlusion time is limited to the time during which blood flow into and out of the limb can safely be interrupted and to the time necessary to control pain felt by the patient during the occlusion. Excessive stagnation of blood within the limb may lead to possible thrombosis, although the occlusion time may be extended with the use of standard anticoagulants such as heparin. The infusion process is repeated as often as desired to provide a desired perfusion of the medicinal agent into the surrounding tissue at the treatment site. Since each dose of the medicinal agent is very small, the systemic buildup of the medicinal agent is minimal, even after several doses have been administered. A typical sequence would be four minutes during which blood flow is occluded and the medicinal agent is infused, followed by releasing the flow restriction for one minute to enable blood to again flow into and out of the limb. The repetitive sequence of occluding the flow of blood, injecting the agent, waiting a specified period of time, and reestablishing the blood flow for a specified period of time may be done manually. However, automatically performing these steps is simpler and more reliable.

Another aspect of the invention is a system for delivering a medicinal agent to a treatment site within a patient. The system includes an infusion catheter and an external constrictor. The system may also preferably include a delivery device for infusing the medicinal agent. An introducer sheath is preferably included and can be inserted into the patient's vein or artery to provide easier and reusable access for inserting the catheter. The catheter is suitable for placement within a vein or artery, and includes at least one infusion lumen extending from an external proximal port to an internal distal port. The catheter may include a radio opaque element disposed adjacent to its distal end to assist in routing the catheter through the vascular system and positioning the distal end of the catheter at the treatment site. To further assist in routing and positioning, the catheter may also include a second lumen adapted to receive a guide wire. The catheter may also optionally further include an enlarged portion adjacent to its distal tip that is adapted to wedge against the inside wall of the vessel to prevent the medicinal agent from flowing away from the treatment site, past the outer surface of the catheter.

The proximal end of the infusion lumen is coupled in fluid communication with an outlet orifice of an infusion delivery device for controlled infusion of a therapeutic drug or diagnostic agent. This infusion delivery device can be a syringe pump, a drug infusion pump, or a similar drug delivery pump. The delivery device may also include a sensor for measuring a quantity of the medicinal agent delivered. The system also preferably comprises an external constrictor that applies pressure to an external portion of the limb, between the patient's heart and the treatment site, to occlude the outflow of blood from the treatment site. The constrictor preferably includes a pressure cuff that is inflated with an inflation pump, or alternatively, comprises a tourniquet adapted to wrap around a limb of the patient and to apply compression to the limb. Optionally, a pressure sensor is included for measuring the pressure provided by the inflation pump, if the pressure cuff is employed.

An automated embodiment of the system includes a controller connected to the infusion delivery device to regulate the flow of medicinal agent to the catheter and to the constrictor, and to control pressurization of the constrictor. One form of the controller includes simple timers that determine time intervals for energizing the drug infusion delivery device and a pressurized fluid source that pressurizes the constrictor, while another form of the controller has a processor that executes machine instructions to control the repetitive constriction and drug infusion process. Preferably, the controller implements a rest/pressure timer function to determine a rest period (the period during which the pressure applied to the constrictor is released) and a pressurization period, to automatically pressurize and release the pressure applied to the constrictor at predetermined intervals. The controller also preferably automatically determines when the delivery device should be activated and deactivated. For example, the delivery device can be activated at the same time that the constrictor is activated, or after a predetermined delay following activation of the constrictor, or when a predetermined pressure has been applied by the constrictor. The controller may further automatically activate the delivery device for a predetermined dosage time period or until a predetermined dose of the medicinal agent has been delivered.

Another aspect of the invention is directed to a method for controlling delivery of a medicinal agent to a treatment site within a limb of a patient through a lumen of a catheter that has been inserted into a blood vessel of the patient and advanced to the treatment site. The method comprises the steps of activating a constrictor that applies an external pressure to stop the flow of blood within the limb in which the treatment site is disposed, and activating a delivery device that is adapted to deliver the medicinal agent to the treatment site through the catheter to keep the medicinal agent at the treatment site at least while the blood flow in the limb is stopped. Preferably, the constrictor is automatically activated for a predetermined constriction period to stop the flow of blood while the delivery device is automatically activated for a predetermined infusion period or as necessary to infuse a predetermined dose of the medicinal agent. The delivery device may be deactivated if a total quantity of the medicinal agent delivered equals a predetermined limit. After the medicinal agent is delivered, the method further includes the steps of deactivating the constrictor to allow blood flow to resume in the limb and the treatment site during a rest period. Following the rest period, the above steps are optionally repeated for a desired number of cycles.

Another aspect of the invention is directed to a machine readable medium on which are stored machine readable instructions, which when executed by a processor, cause it to perform functions generally consistent with the steps described above.

Yet another aspect of the invention is directed to safe and effective methods and systems for localized delivery of therapeutic or diagnostic agents into a desired tissue location that includes a capillary system within an interstitial tissue. This aspect of the invention is specifically directed to a method and system that will provide for low volume, localized delivery of medications directly into capillaries and the surrounding interstitial tissue in mid locations of limbs with minimal distribution of medications to other regions of the patient's body.

In one embodiment, a drug delivery catheter is inserted into the patient's vascular system, either a vein or artery, and advanced peripherally to the desired tissue location within a limb. Methods for passing the catheter in a retrograde direction in veins containing valves are described in U.S. patent application Ser. No. 09/595853 “METHODS OF CATHETER POSITIONING AND DRUG DELIVERY IN VEINS CONTAINING VALVES”, the disclosure and drawings of which are hereby incorporated by reference. Alternatively, a catheter may be placed within a vein in a distal portion of the limb. One preferred location in the leg would be the posterior tibial vein located below the calf. Once introduced into an appropriate vein, the distal tip of the catheter is advanced within the desired tissue location. In order to prevent loss of the medical agent into other regions of the patient, fluid flow away from the desired tissue location must be stopped. An occlusion means, placed proximal to the desired tissue location prevents fluid flow proximally, toward the heart, through the arteries, veins, and lymph vessels.

As noted above, even though only a fraction of the patient's total tissue mass may be located distal to the desired tissue location, it is nonetheless desirable to prevent the medical agent from flowing into this distal area. Thus, one aspect of the present invention involves the use of second occlusion means distal to the desired tissue location, to prevent tissue distal to the treatment site from receiving medical agents introduced into the treatment site.

Preferably, the occlusion means comprise a tourniquet, pressure-cuff, or similar other external device for interrupting blood flow with pressure applied to a limb. After the desired tissue location has been isolated by the occlusion means, a small quantity of the therapeutic or diagnostic agent is injected through the delivery catheter. Since the desired drug delivery location is isolated by the occlusion means, only a small amount need be injected into the vascular system in the desired location. As is known in the art, higher pressures within the capillaries and venules will increase the transfer of the medical agent across the vessel walls and into the interstitial space and tissues surrounding the vessels. Additionally, many medications are known to dilate and further separate the endothelial cells, thereby increasing the transfer of medicinal agents across the vessel walls. One such medicinal agent is papaverine. Such transfer enhancing medications may be administered either before, during, or after administration of the therapeutic agent and can be very useful in increasing the transfer of large molecules across the endothelium of the capillaries and venules.

After a specific period of time, the occlusion means are released, enabling normal blood and lymph flow through the desired tissue location to resume. The occlusion time is limited to that required to ensure the safe cessation of blood flow and to minimize any pain felt by the patient during the occlusion. It is recognized that excessive stagnation of blood within the limb may lead to possible thrombosis, although the occlusion time may be extended with the use of standard anticoagulants, such as sodium heparin.

Because the therapeutic agent is delivered immediately adjacent to the target area, the total dose of the therapeutic agent is significantly lower than required in traditional therapies. Because of the small dose employed, the systemic buildup is minimal. In order to completely fill the venules and capillaries with the therapeutic agent at the desired tissue location, blood may be first removed from the vascular system within the desired tissue location. The blood removal is accomplished by applying negative pressure to the medical agent delivery catheter after the occlusion devices have been activated on the limb, and withdrawing at least a portion of the blood from the vessels.

In a different embodiment, a third fluid displacement cuff, spaced between the proximal and distal cuffs, is employed to first compress the target regions and then to remove a substantial amount of fluid from the blood and lymph vessels in the target region. The proximal and distal cuffs are then inflated to isolate the desired tissue location, and the fluid displacement cuff is released. Thereafter, the therapeutic agent is administered within the desired tissue location. After a suitable time has elapsed, i.e., sufficient to ensure that transfer of the medical agent across the walls of the capillaries and venules and into the surrounding tissues, the fluid displacement cuff is reinflated to displace any residual medical agent back into the administration catheter from the vessels within the desired tissue location. This step further reduces the amount of therapeutic agent that is released into other parts of the patient's body.

The sequence of activating the fluid displacement cuff, activating the occlusion means, deactivating fluid displacement cuff, injecting the agent, waiting a specified period of time, reactivating the fluid displacement cuff and then deactivating the fluid displacement cuff and occlusion means to reestablish the blood flow can be performed manually. Preferably, the procedure is automated to provide a simpler and more reliable method of treatment. Therefore, yet another embodiment of the present invention is an automated system. Such a system includes a catheter suitable for placement within a vein or artery and having at least one infusion lumen extending from about the proximal end to about the distal end. Placement of the catheter in a vein is generally preferred because of the many interconnecting branches that lead to the capillary beds within a desired tissue location. It should be noted however, that placement of the catheter within an artery will accomplish similar results if the catheter is placed within a branch that leads to a capillary bed within the desired tissue location.

Another aspect of the present invention is directed to a system for carrying out the steps of the method described above. The system minimally includes a catheter and external occlusion means. Preferably the system includes fluid infusion means, coupled to the proximal end of the catheter for controlled infusion of a therapeutic or diagnostic agent. Such fluid infusion means comprise a syringe pump, a drug infusion pump, a solution bag maintained at an appropriate height above the infusion site, a solution bag contained within a pressurizing cuff, or the like.

In one embodiment, the occlusion means comprises a tourniquet. More preferably, the occlusion means comprises a pressure activated cuff. In one embodiment, two external pressure cuffs are provided, with one cuff disposed proximal to the treatment site to stop blood flow into the limb, and the second cuff disposed distal to the treatment site to prevent an infused medical agent from flowing into tissue distal of the treatment site. A fluid displacement cuff is optionally included and employed to remove a substantial amount of fluid from the treatment site before the proximal and distal cuffs are activated. A single inflation pump can be provided to control all cuffs, or each cuff can be coupled to a separate inflation pump. Alternatively, a single pump can control the fluid displacement cuff, and a different inflation pump can control both the proximal and distal cuffs. If a single inflation pump is employed with a fluid displacement cuff, a valve is employed to enable either the displacement cuff or the proximal and distal cuffs to be selected. Preferably, such a system includes a bleed valve coupled to the fluid displacement cuff, so that it may be deflated without deflating the proximal and distal cuffs.

In another embodiment, only one pressure cuff (substantially larger than the treatment site) is employed, such that this pressure cuff overlaps the treatment site, extending beyond the treatment site in both the proximal and distal directions.

Preferably, the present invention also includes a control system for controlling the fluid infusion flow rate, delivery pressure and start and stop times, and occlusion means start and stop times. One embodiment of the control system is relatively simple, comprising timers that control the drug infusion means, the occlusion means, and the fluid displacement cuff, while a more sophisticated control system includes a programmed processor.

In one embodiment, the infusion catheter contains a second lumen extending from the proximal end to the distal end, suitable for receiving a guide wire used for navigation through the vasculature in a patient's body.

### BRIEF DESCRIPTION OF THE DRAWING FIGS.

The foregoing aspects and many of the attendant advantages of this invention will become more readily appreciated as the same becomes better understood by reference to the following detailed description, when taken in conjunction with the accompanying drawings, wherein:

FIG. 1 is a schematic view of a portion of a patient's leg and foot, showing a catheter inserted into a vein and a flow restricting cuff applied to the leg, in accord with the present invention;

FIG. 1A is an enlarged cross-sectional view of the catheter of FIG. 1;

FIG. 2 is a schematic view of a portion of a patient's leg and foot, illustrating a block diagram of a system for localized drug delivery through an artery to a treatment site in the foot, in accord with the present invention;

FIG. 3 is a block diagram of a controller for automatically providing repetitive delivery of a medicinal agent to a treatment site in a limb of the patient;

FIG. 4 is a block diagram of a programmed processor-based controller for automatically providing repetitive delivery of a medicinal agent to a treatment site in a limb of the patient;

FIG. 5 is a flow chart of the control logic implemented by the controller of FIG. 4, to control the repetitive delivery of medicinal agent to the treatment site;

FIG. 6 is a schematic illustration of a portion of a patient's leg and foot, showing a catheter inserted into a vein and two flow restricting cuffs applied to the leg, distal and proximal of a treatment site, the proximal flow restricting cuff stopping blood flow in the leg, and the distal flow restricting cuff preventing any medicinal agent delivered to the treatment site from migrating to tissue distal of the treatment site;

FIG. 7 is a schematic illustration of a portion of a patient's leg with a balloon catheter inserted into an artery and a flow restricting cuff applied to the leg, distal of a treatment site, the flow restricting cuff preventing any medicinal agent delivered to the treatment site from migrating to tissue distal of the treatment site, and the balloon inflated to prevent any medicinal agent from flowing away from the treatment site in a proximal direction;

FIG. 8 is the schematic illustration of FIG. 6, further incorporating a fluid displacement cuff;

FIG. 9 is a schematic illustration of a portion of a patient's leg and foot, with a single relatively larger flow restricting cuff applied to the leg;

FIG. 10A is a block diagram of an automated system for providing localized drug delivery according to the illustration in FIG. 6;

FIG. 10B is a block diagram of an automated system for providing localized drug delivery according to the illustration in FIG. 6, with a valve that selectively determines an order in which the proximal and distal flow restriction cuffs are inflated;

FIG. 10C is a block diagram of an automated system for localized drug delivery according to the illustration in FIG. 7;

FIG. 10D is a block diagram of an automated system for localized drug delivery according to the illustration in FIG. 8, wherein each flow restriction cuff and the fluid displacement cuff are activated by an individual inflation pump;

FIG. 10E is a block diagram of an automated system for localized drug delivery according to the illustration in FIG. 8, wherein both flow restriction cuffs are activated by an individual inflation pump, and the fluid displacement cuff is activated by an individual inflation pump; FIG. 10F is a block diagram of an automated system for localized drug delivery according to the illustration in FIG. 8, wherein both flow restriction cuffs and the fluid displacement cuff are activated by an individual inflation pump;

FIG. 10G is a block diagram of an automated system for localized drug delivery according to the illustration in FIG. 9;

FIG. 11A is a flow chart of the control logic implemented by a controller to control the repetitive delivery of medicinal agent to the treatment site using the automated system of FIGS. 10A and 10G;

FIG. 11B is a flow chart of the control logic implemented by a controller to control the repetitive delivery of medicinal agent to the treatment site using the automated system of FIG. 10B;

FIG. 11C is a flow chart of the control logic implemented by a controller to control the repetitive delivery of medicinal agent to the treatment site using the automated system of FIG. 10C;

FIG. 11D is a flow chart of the control logic implemented by a controller to control the repetitive delivery of medicinal agent to the treatment site using the automated system of FIG. 10D;

FIG. 11E is a flow chart of the control logic implemented by a controller to control the repetitive delivery of medicinal agent to the treatment site using the automated system of FIG. 10E; and

FIG. 11F is a flow chart of the control logic implemented by a controller to control the repetitive delivery of medicinal agent to the treatment site using the automated system of FIG. 10F.

### DESCRIPTION OF THE PREFERRED EMBODIMENTS

The drawings illustrate both the design and utility of several preferred embodiments of the present invention. Similar elements of the different embodiments are identified with the same reference numbers to simplify the description of each preferred embodiment. As used herein, the term “medicinal agent” refers to any therapeutic agent or any diagnostic agent that might be infused to an internal treatment site within a limb of a patient's body. The term “therapeutic agent” refers to any chemical, biological, or other material that is used in the treatment of a disease or disorder. Examples, without limitation, of therapeutic agents are antibiotics, chemotherapy agents, gene therapy agents, anti-neoplastics, hormones, antivirals, radiation sources (such as cobalt, radium, radioactive sodium iodide, etc.), anticoagulants, enzymes, hepatoprotectants, vasodilators, prodrugs, and the like. Any therapeutic agent that is a liquid, or can be dissolved in a liquid, or carried in suspension by a liquid may be administered using the present invention.

As used herein, the term “diagnostic agent” refers to any chemical or other material that is used to determine the nature of a disease or disorder. Examples, without limitation, of diagnostic agents are dyes that react with metabolic products of a particular disease, and radioactive materials that bind to and thereby indicate the presence of disease-causing entities within a patient's body. As is the case with therapeutic agents, any diagnostic agent that is a fluid, or can be dissolved in a fluid, or carried in suspension by a fluid may be employed using the devices and methods herein.

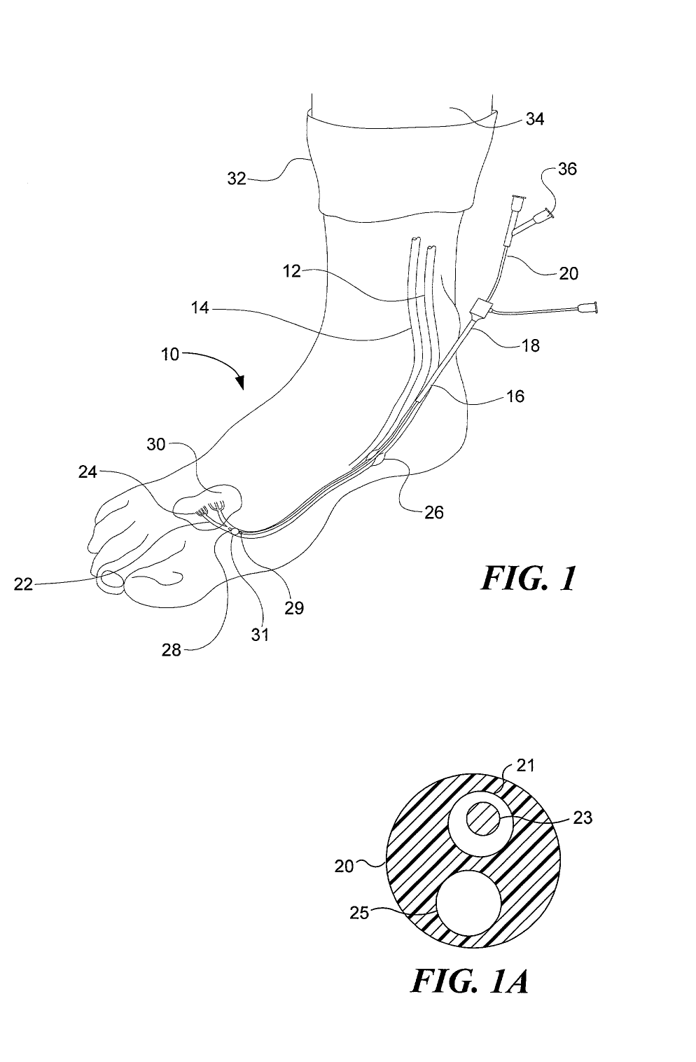

FIG. 1 illustrates a first preferred embodiment of the present invention as used to infuse a medicinal agent to a treatment site within a foot 10 of a patient. Within foot 10 are generally parallel extending veins 12 and 14 that drain blood from the lower leg and foot. Inserted into vein 12 at a puncture site 16 is an introducer sheath 18 that facilitates insertion of a catheter 20, which delivers a medicinal agent to a treatment site 30. The medicinal agent is to be delivered to the treatment site disposed proximate the end of vein 12, where the vein divides into smaller venules 22, that further subdivide into capillary vessels 24. To reach this location, catheter 20 is guided through introducer sheath 18 and advanced retrograde through a venous valve 26 within vein 12 until a distal tip 28 of the catheter is disposed adjacent to treatment site 30.

As shown in FIG. 1A, catheter 20 includes a lumen 21 through which a guide wire 23 may be inserted to assist in guiding the catheter through the vasculature system of the patient, to a desired position within a vessel. Also included is a lumen 25 for use in infusing a medicinal fluid. Catheter distal tip 28 may optionally include a radio-opaque element 29 that is readily visible in X-ray images, to assist in advancing and positioning the catheter distal tip adjacent to the treatment site. Additionally, details of the steps involved in advancing a catheter in a retrograde direction within veins containing valves are described in commonly assigned U.S. patent application, Ser. No. 09/595,853, entitled METHODS OF CATHETER POSITIONING AND DRUG DELIVERY IN VEINS CONTAINING VALVES filed on Jun. 16, 2000, the drawings and specification of which are hereby specifically incorporated herein by reference.

Referring again to FIG. 1, once distal tip 28 is positioned in the desired location adjacent to the treatment site, blood flow within the foot is stopped using a flow restricting cuff 32 that is placed around a calf 34 on the patient's leg. The flow restricting cuff preferably comprises a conventional tourniquet, or more preferably, comprises an elastomeric annular chamber that is secured around the limb of the patient and then pneumatically inflated—either manually with a squeeze bulb pump (not shown), or automatically with a pneumatic pump (not shown) that is electrically energized. Sufficient constriction force is exerted by the flow restricting cuff (or tourniquet) to interrupt blood flow into and out of the limb on which the constricting device is fastened. The flow restricting cuff is similar to the cuff typically applied to a patient's upper arm by medical practitioners when measuring blood pressure. When blood flow within the foot (i.e., flow into and out of the foot) has been stopped by the flow restricting cuff, a medicinal agent (not shown) is administered to the treatment site through lumen 25 of catheter 20, by forcibly injecting the medicinal agent through a Luer fitting 36 that is in fluid communication with the lumen. An enlarged spherical portion 31 of the catheter is formed adjacent to distal tip 28 to seal against the interior surface of the blood vessel and block the medicinal agent from flowing back past the external surface of the catheter within the blood vessel in which the catheter is disposed. Because the blood flow within foot 10 has been stopped, and because the drug is delivered at a precise location adjacent to the treatment site, more of the drug is absorbed by the target tissue at the treatment site than if the drug were externally injected into foot 10 or into the vascular system of the patient. Also, the medicinal agent is administered only to the treatment site. Any concern about the toxicity or other adverse effect of the medicinal agent on the patient's body is minimized, since the medicinal agent has been infused at the treatment site, where its desired action is required and because it will not be conveyed by blood throughout the patient's systemic system, at least until after it has completed its intended function. A relatively small amount of the medicinal agent can be used when infused directly into the treatment site, compared to the much larger dose that would be required if the medicinal agent were simply injected into the patient's body or if administered orally. Accordingly, any toxic or other adverse effects of the medicinal agent on the patient's body are substantially avoided by the present invention as a result of the relatively low dosage of the medicinal fluid required.

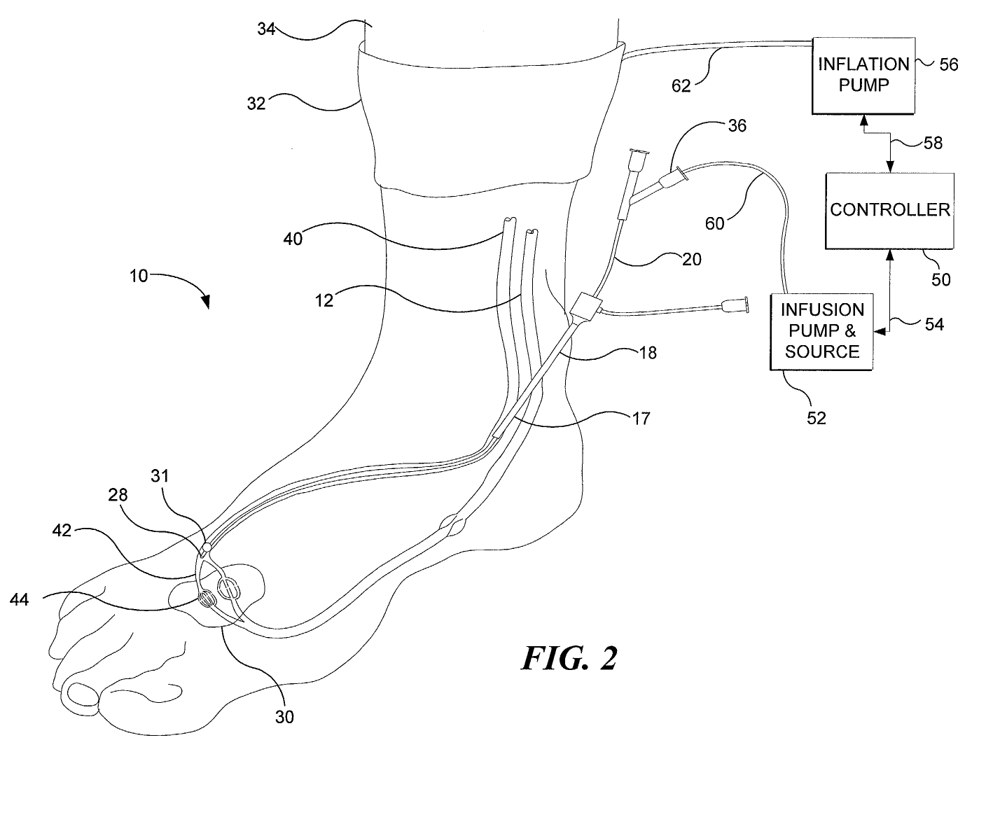

Turning to FIG. 2, a similar preferred embodiment of the present invention is shown in which catheter 20 is advanced antegrade to treatment site 30 through an artery 40 instead of vein 12. Introducer sheath 18 has been inserted into artery 40 at a puncture site 17, facilitating insertion of catheter 20. Proceeding distally down the limb of the patient, artery 40 divides into smaller arterioles 42, which still more distally, divide into capillary vessels 44. Catheter 20 has been advanced antegrade down artery 40, until catheter distal tip 28 is at treatment site 30. Flow restricting cuff 32 is again disposed around calf 34, to obstruct the flow of blood into and out of foot 10 while the medicinal fluid is being administered to the treatment site through catheter 20.

A controller 50 that can automatically control blood flow into and out of the affected limb and administer a medicinal agent to the treatment site in the limb at predetermined intervals of time is applicable to both the venous and arterial applications of the present invention shown respectively in FIGS. 1 and 2. While a conventional personal computer or other general programmed processor (not shown) can be used for controlling and automating the repetitive infusion of the medicinal agent through catheter 20 and controlling the pressurization of cuff 32, it is likely that controller 50 will be specifically designed for this purpose. The controller may be energized with a battery supply (not shown) or by an internal alternating current (ac) line power supply (not shown) or an external conventional ac line transformer “power brick” of the type commonly used to provide power to computer peripheral devices. Controller 50 controls an infusion pump and source 52 via signals conveyed over an infusion control line 54 and controls an inflation pump 56 via signals conveyed over an inflation control line 58.

Luer fitting 36 on catheter 20 is coupled in fluid communication with infusion pump and source 52 through an infusion line 60. The infusion pump and source includes a small reservoir, vial, or other container (not separately shown) in which the medicinal agent is stored. When the medicinal agent is administered manually, a conventional syringe can be used to force the medicinal fluid through the catheter to the treatment site. In the automated embodiment shown in FIG. 2, infusion pump 52 preferably comprises an automated syringe pump, a cassette pump, a peristaltic pump, or other suitable medicinal fluid pump that is controlled in response to a signal received from controller 50 over infusion control line 54. Inflation pump 56 is connected in fluid communication with flow restricting cuff 32 via a flexible tube 62 and preferably comprises a standard pneumatic inflation pump of a size and volumetric rating suitable for pneumatically inflating flow restricting cuff 32 to a pressure sufficient to substantially stop blood flow into and out of a limb of a patient, in response to a signal received from controller 50 over inflation control line 58.

After the system has been configured as shown, flow restricting cuff 32 is inflated to stop blood flow into and out of the limb for a specific (predefined) length of time. Once the inflation pressure is sufficient to stop blood flow in the limb, infusion of the medicinal agent (not shown) commences with the delivery of a specific bolus of the medicinal agent by infusion pump and source 52 through line 60 and catheter 20, to treatment site 30. After a predetermined infusion time period has elapsed, the controller causes inflation pump 56 to release the pressure in flow restrictive cuff 32, and blood flow is restored to the leg and foot of the patient. After a specific rest period, the inflation and infusion sequence is repeated by controller 50. In this manner, small doses of medicinal agent are repetitively safely infused into the treatment site.

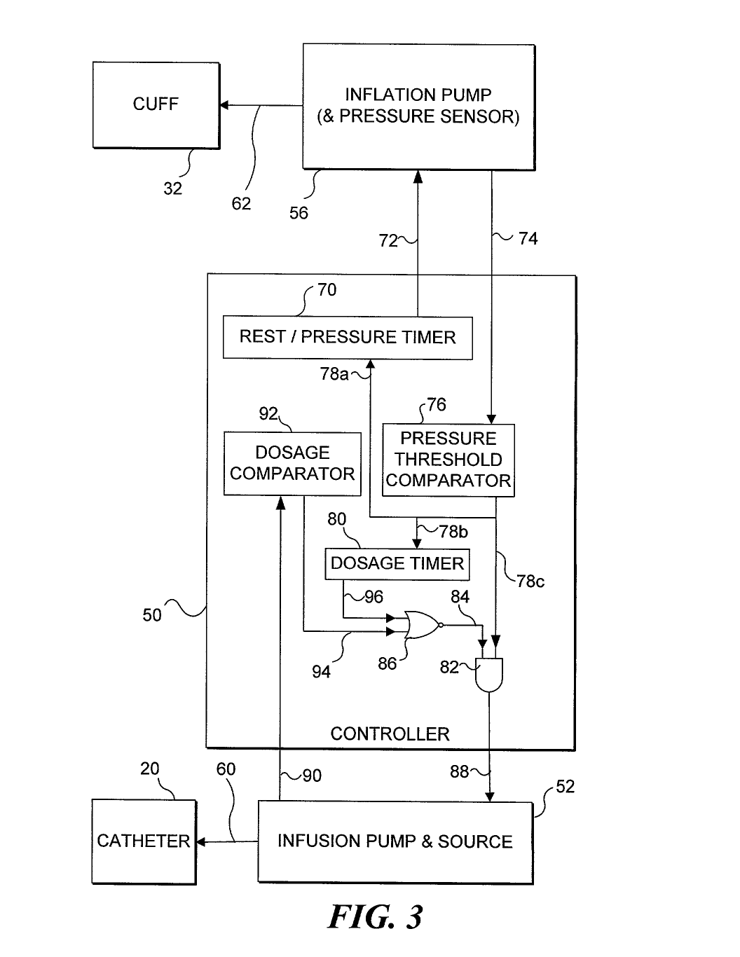

FIG. 3 is a block diagram of a preferred embodiment of controller 50 for providing the repetitive delivery of a medicinal agent in the manner described above. A rest/pressure timer 70 provides an inflate/deflate signal over a line 72 to inflation pump 56 to control the pneumatic pressure in tube 62 and flow restrictive cuff 32. The inflate/deflate signal can optionally be used to start a delay timer (not shown), to provide a delay after starting to pressurize flow restrictive cuff 32, before activating infusion pump 52. Alternatively, the infusion pump can be activated directly by the signal that starts the pressurization of the flow restrictive cuff, if the infusion pump is designed to infuse the medicinal agent sufficiently slowly that the flow of blood in the limb is substantially interrupted before any significant amount of the medicinal agent is infused into the treatment site. Preferably, however, a pressure sensor (not separately shown) in inflation pump 56 returns a pressure signal on a line 74 that is indicative of the pneumatic pressure applied to flow restrictive cuff 32. The pressure signal on line 74 is supplied to a pressure threshold comparator 76, which determines when the pressure in the flow restrictive cuff has reached a threshold level that has been determined to be sufficient to stop the flow of blood in the limb of the patient. When the pressure threshold is reached, pressure threshold comparator 76 provides a full-pressure signal on a line 78a, which is coupled to rest/pressure timer 70, and the rest/pressure timer determines the time that inflation pump 56 maintains this pressure within flow restrictive cuff 32. The time interval for maintaining the pressure is preferably about ten minutes. The full-pressure signal is also provided over a line 78b to a dosage timer 80, which determines the time interval for infusing the medicinal agent into the treatment site. This time interval for the infusion of the medicinal agent is thus initiated when the full-pressure signal indicates that the flow of blood in the limb has been stopped.

Pressure threshold comparator 76 also provides the full-pressure signal over a line 78c to an infusion logic gate 82 to indicate that the pressure in the flow restrictive cuff is adequate to stop the flow of blood in the limb, so that the medicinal agent can then be infused into the treatment site in the patient. In this embodiment, infusion logic gate 82 is an AND gate with two inputs and one output. Infusion logic gate 82 also receives a full-dosage signal on a line 84 from a dosage logic gate 86. Dosage logic gate 86 is a NOR gate providing an inverse full-dosage signal on line 84. Until the full dosage is reached, the logic level of the full-dosage signal on line 84 remains high, enabling infusion logic gate 82 to toggle, based on the full-pressure signal on line 78c. When the logic level of the full-pressure signal on line 78c is high, infusion logic gate 82 provides a high logic level infusion signal on a line 88 to infusion pump 52, enabling it to be energized. Those of ordinary skill in the art will realize that many different logic circuits and components may be combined to achieve a comparable result.

When infusion signal 88 is high, infusion pump 52 delivers the drug through tube 60 to catheter 20, which is routed to the treatment site. A total flow transducer (not shown) in infusion pump 52 returns a flow signal on a line 90 that is indicative of the quantity of medicinal agent delivered to the treatment site through catheter 20. A dosage threshold comparator 92 determines when the dose of medicinal agent delivered to the treatment site is equal to a predetermined level for one cycle of infusion. Dosage threshold comparator 92 can be used to control the infusion of a desired quantity of the medicinal agent during each infusion cycle, as a metering device with a variable setting to regulate the flow rate of the medicinal agent administered to the treatment site to a desired level for a predetermined time, or as an emergency shut-off, to prevent an excessive quantity of the medicinal agent from being administered. When the predetermined dosage level or threshold is reached, dosage threshold comparator 92 provides a full-quantity signal on a line 94 to infusion logic gate 82. A high logic level full-quantity signal on line 94 causes the output of dosage logic gate 86 to go low, which causes the output of infusion logic gate 82 to go low, stopping infusion pump 52 from delivering any more of the medicinal agent to the treatment site.

Similarly, when dosage timer 80 determines that the time interval during which the medicinal fluid is to be administered has elapsed, the dosage timer provides a dosage time-out signal on a line 96 to infusion logic gate 82. The drug delivery time interval is preferably the same as the pressure duration, i.e., approximately ten minutes. A high logic level dosage time-out signal on line 96 also causes the output of dosage logic gate 86 to go low, which causes the output of infusion logic gate 82 to go low, stopping infusion pump 52 from delivering any more of the medicinal agent.

When an infusion period is complete, rest/pressure timer 70 provides a signal over line 72 to inflation pump 56 that causes the inflation pump to release the pressure in flow restricting cuff 32, so that blood flow in the limb of the patient resumes. Rest/pressure timer 70 then initiates a predetermined rest period, and after the rest period is complete, begins the pressurization and infusion cycle again. Controller 50 may also include a cycle counter (not shown) that counts the cycles until a desired number of cycles of medicinal fluid infusion have been achieved, causing the repetitive process of medicinal fluid infusion to be stopped.

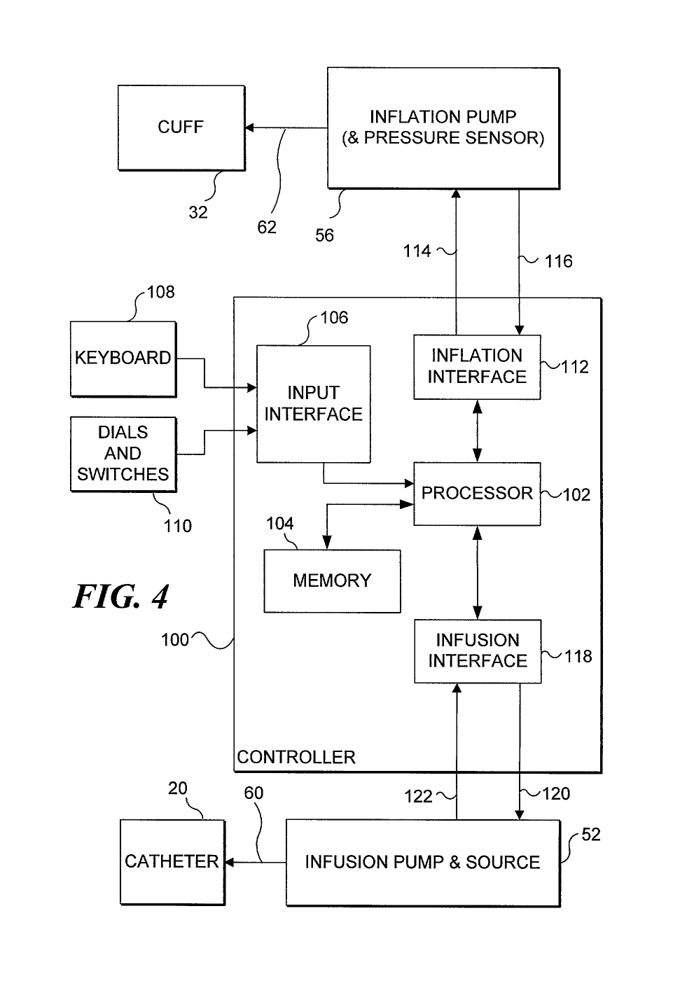

FIG. 4 illustrates a processor-based controller 100 that automates the delivery of a medicinal agent to a treatment site. Controller 100 may be a specialized device designed specifically for the purpose of controlling delivery of the medicinal agent to a treatment site, or a general computing device, such as a personal computer that is programmed to do so. Controller 100 includes a processor 102, which may be a microcontroller if controller 100 is a specialized device, or may be a typical processor of the type commonly used in a personal computer. Processor 102 is coupled to a memory 104 in which machine instructions and data are stored. Memory 104 includes volatile random access memory (RAM) and non-volatile read only memory (ROM). Controller 100 may also include a permanent storage (not shown), such as a hard disk, and a removable storage medium drive (not shown), such as a floppy disk drive. Also connected to processor 102 is an input interface 106, which provides communication with a keyboard 108. The keyboard may be a specialized keypad with control specific functional buttons, or a general purpose computer keyboard. Keyboard 108 is used to enter commands and parameters for controlling delivery of a medicinal agent to a treatment site. Displays and switches 110 are additionally or alternatively used to enter commands and parameters to control delivery of the medicinal agent. For example, displays and switches 110 may be used to manually enter a constriction time period and a rest period, and to specify a number of cycles, a dosage of the medicinal agent per cycle, a maximum total allowable dosage, and the like.

Processor 102 also communicates with an inflation interface 112, sending activation and deactivation commands to the inflation interface, and receiving pressure data from it. Inflation interface 112 optionally includes an analog-to-digital converter (ADC) (not separately shown) for converting the analog pressure signals produced by a pressure sensor included in inflation pump 56 to corresponding digital pressure data. The processor produces a command signal 114 to control inflation pump 56. As explained above, inflation pump 56 provides pressurized air through tube 62 to cuff 32 to inflate the cuff sufficiently to constrict the flow of blood in the limb of a patient.

In addition, processor 102 communicates with an infusion interface 118, sending activation and deactivation commands, and receiving medicinal agent flow data. Infusion interface 118 sends a command signal 120 to infusion pump 52 to activate and deactivate the infusion pump. An ADC (not shown) is provided for receiving an analog flow signal 122 produced by a flow sensor (not shown) in the infusion pump and converting the analog signal to digital data. As explained above, infusion pump 52 delivers the medicinal agent to the treatment site in the limb of the patient through tube 60 and through catheter 20. Infusion pump 52 may also include a reservoir, vial, or other source (not separately shown) for the medicinal agent, or may comprise a motorized syringe that simply delivers the medicinal agent contained within the syringe by advancing a plunger (not shown).

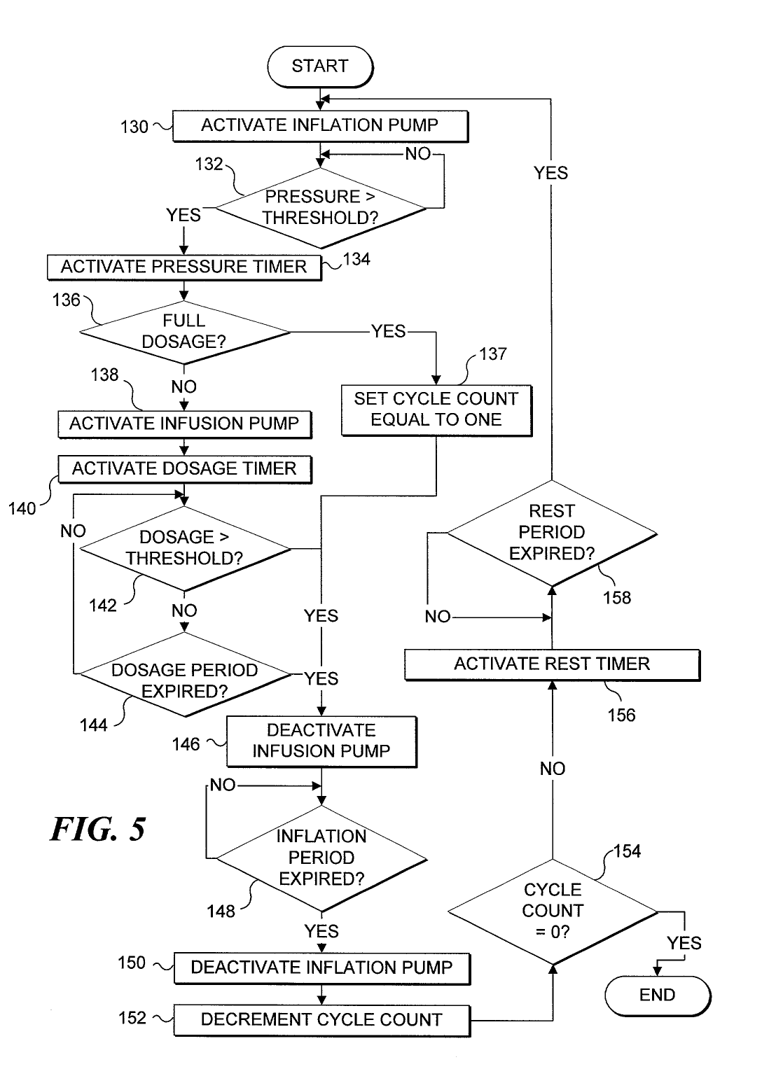

FIG. 5 illustrates the control logic that is implemented in software or in hardwired logic to control repetitive cycles of infusion of a medicinal agent to a treatment site in a limb of a patient. At a block 130, inflation pump 56 is activated to pressurize flow restrictive cuff 32 to stop the flow of blood in the affected limb. At a decision block 132, the pneumatic pressure in the flow restrictive cuff is compared with a predetermined pressure threshold value to determine if the pressure in the flow restrictive cuff is sufficient to stop blood flow in the limb. This step is repeated until the detected pressure value in the flow restrictive cuff is greater than the pressure threshold value. A pressure timer is then activated at a block 134 to begin a predetermined inflation period during which the flow of blood in the limb is stopped.

A decision block 136 determines if a predetermined total dosage of the medicinal agent has been administered. The amount of medicinal agent administered each cycle is determined by a flow transducer in the infusion pump or other sensor and compared with a predetermined total dosage value that can be set by a medical practitioner as a desired dosage or as a maximum allowable dosage. If a full dosage of the drug has already been administered so that no further drug infusion is required by the infusion pump, then the cycle count is set to one in a block 137 and the infusion pump is deactivated at a block 146. The logic can be modified so that a full dosage result will also deactivate the inflation pump, as indicated in a block 150, causing blood flow in the limb to be immediately enabled. However, the logic shown maintains the pressure in the flow restrictive cuff for the entire inflation period so that any manually administered drug may be perfused into the tissue of the treatment site without blood flow carrying away the drug.

If a full dosage is not detected, the infusion pump is activated in a block 138, and a dosage timer is activated in a block 140. A decision block 142 determines whether the quantity of the medicinal agent delivered during a current cycle has reached a desired dosage threshold. If so, then the infusion pump is deactivated in block 146. If the dosage threshold for the current cycle has not been reached, a decision block 144 determines whether a dosage period has expired, as established by the dosage timer. If the dosage period has not yet expired, the drug delivered is checked again at decision block 142. When the dosage period has expired, the infusion pump is deactivated at block 146.

A decision block 148 then determines whether a current inflation period has expired, as determined by the pressure timer. Until the inflation period has elapsed, the logic loops, providing time for the delivered drug to perfuse the tissue of the treatment site while the blood flow is stopped. Once the inflation period has expired, the inflation pump is deactivated at a block 150.

Block 150 concludes a cycle of medicinal agent infusion, so a cycle counter is decremented in a block 152. A decision block 154 then determines whether all of a predetermined number of cycles of infusion of the medicinal agent have been completed. If all of the infusion cycles have been completed, the process ends. If additional infusion cycles remain, the rest timer is activated in a block 156. A decision block 158 then determines whether the rest period has expired, as determined by the rest timer. Until the rest period has elapsed, the logic loops, providing time for blood flowing in the limb and treatment site to re-oxygenate tissue in the portion of the limb where blood flow was previously interrupted. Once the rest period has expired, the next infusion cycle begins by reactivating the inflation pump at step 130.

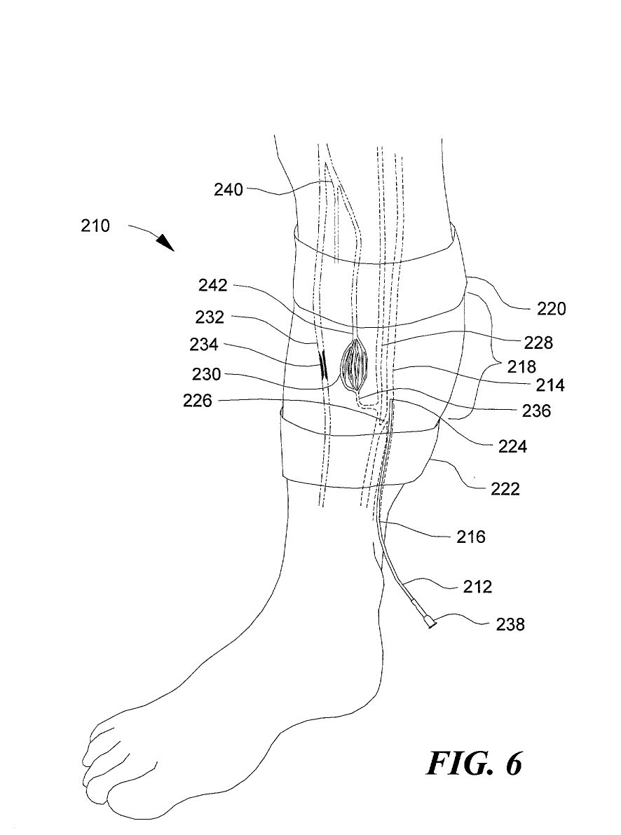

Yet another embodiment of the present invention enables a therapeutic agent to be delivered to a target area in a limb of a patient, without delivering the therapeutic agent to non target locations within the limb or to other portions of the patient's body. FIG. 6 illustrates a delivery catheter 212 inserted into a vein 214 in a patient's leg 210 at a puncture site 216. Catheter 212 has a continuous lumen (not shown) that extends from a distal end 224 and terminates at a medical Luer fitting 238 disposed at its proximal end. A medical agent container and pressurizing means (not shown) such as a syringe pump, drug infusion pump, or a pressurized bag, is attached to Luer fitting 238. Placement of the catheter in a vein is generally preferred because of the many interconnecting branches that lead to the capillary beds within the desired tissue location. It should be noted, however, that placement of the catheter within an artery will accomplish similar results if placed within a branch that leads to a capillary bed within the desired tissue location. A desired tissue location 218 is isolated from the rest of the patient's body by the external placement of a proximal flow restricting cuff 220 and a distal flow restricting cuff 222. Note that these occlusion devices are external to vein 214, and are disposed around leg 210 of the patient.

As shown in FIG. 6, distal end 224 of catheter 212 has been advanced to a target area within desired tissue location 218. As those of ordinary skill in the art will recognize, although not shown in this figure, the proximal end of the catheter is coupled via Luer fitting 238, to the outlet orifice of fluid infusion means that provide controlled infusion of a therapeutic or diagnostic agent, as noted above. Vein 214 is in fluid communication with additional veins 228 within tissue location 218 via interconnecting vein branches 226. A venule 236 drains a capillary system 230 that is adjacent to an artery 232, which passes through desired tissue location 218 and which contains a flow restricting stenosis 234. The present invention is thus used to treat flow restricting stenosis 234, to increase blood flow to the lower part of leg 210. Preferably any medical agent introduced into the area immediately adjacent to flow restricting stenosis 234 will be prevented from flowing up the leg via veins or down the leg via arteries. In this embodiment of the present invention, external occlusion devices are employed to prevent such migration away from the desired treatment site. Any flow of the medical agent up the leg via arteries, such as artery 232 or artery 240, via veins such as vein 214 or vein 228, or via any lymph vessels (not shown) is prevented by activating proximal flow restricting cuff 220. Similarly, any flow of the medical agent down the leg via arteries, veins or lymph vessels is prevented by activating distal flow restricting cuff 222.

The external occlusion devices may alternatively comprise conventional tourniquets, but more preferably, the pressure cuffs are employed, generally as described in regard to the embodiment of FIG. 2. The pressure cuffs, as described above, generally each comprises an elastomeric annular chamber that is secured around the limb of the patient and then pneumatically inflated, either manually with a squeeze bulb pump (not shown), or automatically with a pneumatic pump that is electrically energized (see FIG. 2). Sufficient constriction force is thus exerted by the flow restricting cuff (or tourniquet) to interrupt blood flow into and out of the limb on which the constricting device is fastened. The simultaneous use of both proximal flow restricting cuff 220 and distal flow restricting cuff 222 enables a desired portion of a limb (leg or arm) to be isolated from the patient's circulatory system, ensuring that any medicinal agent delivered to the desired area does not migrate to another portion of the patient's body. By concentrating the medicinal agent only at the area it is required, less of the medicinal agent is required, and deleterious side effects to non-target tissue are minimized.

The general manner in which the proximal and distal flow restricting cuffs, and the catheter illustrated in FIG. 6 are employed is as follows. The catheter is inserted into the vein or artery selected and is advanced to the desired location (i.e., adjacent to a treatment site, such as stenosis 234). The proximal flow restricting cuff and the distal flow restricting cuff are located above and below the desired location, thereby determining the extent of the area that will be isolated from the patient's circulatory system. The cuffs are activated, either by tightening a tourniquet or inflating a pressure cuff. The desired medical agent is injected, and the practitioner waits for a specified period of time. The time required is a function of the medical agent employed and the condition to be treated. After the desired time period has elapsed, the proximal flow restricting cuff and the distal flow restricting cuff are deactivated to reestablish the blood flow in the limb of the patient.

Restricting blood flow to a portion of a limb for over 30 minutes is rarely advisable, and the specified time period will preferably be significantly shorter in duration. If treatment is required for a time period longer than 5-10 minutes, the treatment will preferably be provided in a series of short intervals (e.g., each about 5 minutes or less) spread out over a long period of time. Between each short treatment interval, the flow restricting cuff will be relaxed, enabling normal blood flow to resume. Preferably, the catheter will remain in place until no further treatment is required, to eliminate any risk of injury associated with repetitively implanting a catheter.

In one embodiment of the present invention, a gene therapy medical agent known as a polynucleotide is introduced into desired tissue location 218 through delivery catheter 212 via vein 214, venule 236, and capillary system 230. Note that desired tissue location 218 surrounds stenosis 234 in artery 232. The specific polynucleotide gene therapy medical agent employed might be engineered to express a vascular endothelial growth factor (VEGF) protein. After uptake of the polynucleotide, cells within desired tissue location 218 will express VEGF, thus generating new blood vessels (not shown) that will reduce the undesirable effects of stenosis 234. Catheter 212 may alternately be placed within artery 240 if its distal end 224 can be navigated into artery branch 242, which feeds capillary system 230. Note that positioning distal end 224 of catheter 212 within occluded artery 232 will not deliver the medical agent into desired tissue location 218 because artery 232 does not have any branches that feed a capillary system within desired tissue location 218.

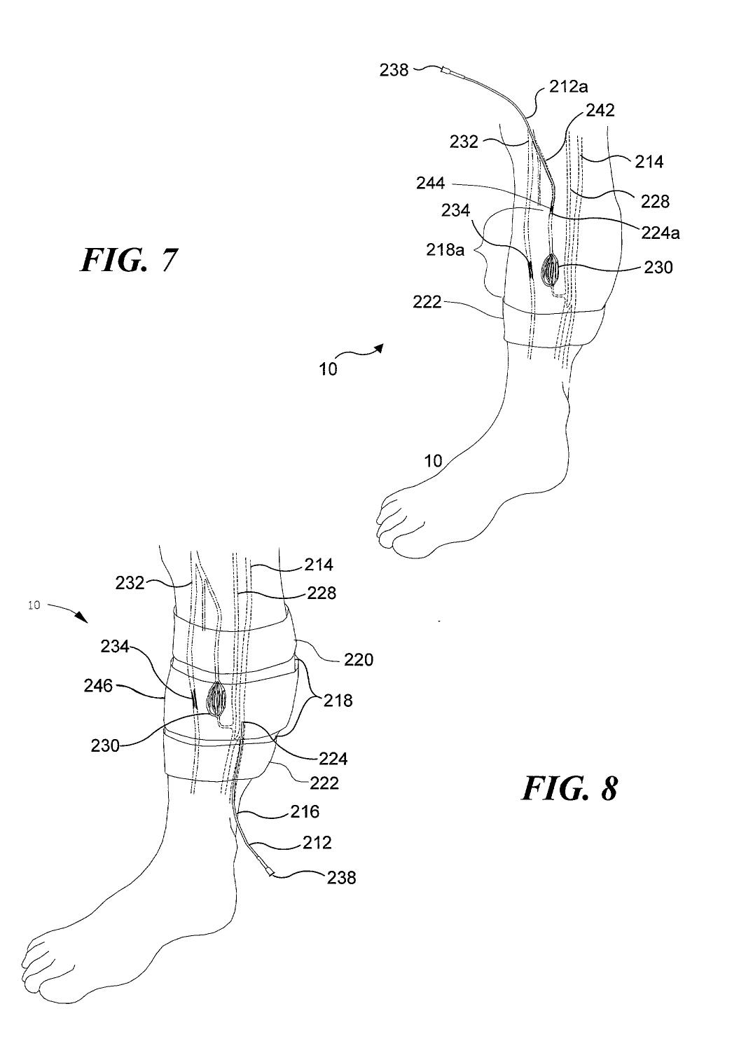

It is anticipated that a desired tissue location can alternatively be isolated using only a single external flow restricting cuff, if a balloon catheter is also employed. In such an embodiment, the region between the balloon portion of the catheter and the external flow restricting cuff can be isolated from the balance of the patient's circulatory system. FIG. 7 illustrates such an embodiment. As described above, distal end 224a of a catheter 212a is advanced to a target area within desired tissue location 218. However, as shown in FIG. 7, catheter 212a is advanced to the target area through artery branch 242. It should be understood that the technique of employing a single external flow restricting cuff and a balloon catheter is not restricted to use only in arteries, as described in this example. The technique could also be employed in veins, such as vein 214, as is shown in FIG. 6. It should also be noted that the position of the single external flow restricting cuff (i.e., either proximal or distal, relative to the balloon) is selected based on the direction of the flow of blood within the vein or artery into which the balloon catheter is inserted. The external flow restricting cuff must be disposed downstream of the balloon and the target site, such that a flow of medical agent downstream of the target site is restricted, thereby isolating the medical agent from the rest of the circulatory system in the patient's body.

Referring once again to FIG. 7, a balloon 244 is incorporated into distal end 224a of catheter 212a. Those of ordinary skill in the art will readily recognize that such inflatable balloons are well known in the art. When inflated, balloon 244 will prevent any medical agent delivered through distal end 224a of catheter 212a to tissue location 218 from diffusing up leg 10 via artery branch 242. The natural flow of blood through artery branch 242 will drive any medical agent released from distal end 224a of catheter 212a towards capillary system 230, which, as noted above, is adjacent to artery 232 in which flow restricting stenosis 234 is disposed. As described above, while passing through capillary system 230, the medical agent will diffuse into the adjacent tissue. When inflated, distal flow restricting cuff 222 prevents the medical agent from flowing down leg 10 past distal flow restricting cuff 222. Thus, balloon 244 and distal flow restricting cuff 222 define tissue location 218 so that it is substantially isolated from the rest of the patient's circulatory system.

It should be noted that some medical agent can diffuse through tissue adjacent to both artery branch 242 and vein 228, entering vein 228 and being carried beyond tissue location 218. While the combination of a balloon catheter and a single external occlusion device significantly reduces the migration of a medical agent beyond tissue location 218, the use of a second external occlusion device (such as proximal flow restricting cuff 220 shown in FIG. 6) is expected to be slightly more effective at reducing migration of a medical agent beyond tissue location 218. However, because using an occlusion device to isolate a portion of a patient's body from a normal blood flow deprives tissue in the affected region from oxygen and nourishment, there are circumstances in which minimizing the area of tissue to which all blood flow is occluded is desirable. As will be evident from FIG. 7, employing distal flow restricting cuff 222 and not proximal flow restricting cuff 220 reduces the region of leg 10 from which normal blood flow is occluded. While balloon catheter 212a can be employed in vein 214 along with proximal flow restricting cuff 220 around leg 10 to similarly isolate tissue location 218, doing so enlarges the portion of the leg through which normal blood flow is occluded. As discussed above, a conventional tourniquet may alternatively be used instead of either flow restricting pressure cuff.

The general manner in which a single proximal or distal flow restricting cuff and the balloon catheter illustrated in FIG. 7 are employed is similar to the method previously described, except that the catheter balloon is inflated to occlude blood flow, in place of the external flow restricting cuff that it replaces. A balloon catheter is inserted into the vein or artery selected, and advanced to the desired location. A single flow restricting cuff is located downstream of the distal end of the catheter. Note that downstream is relative to the direction of the normal flow of blood. If a vein is selected (blood flows to the heart), downstream is a location on the limb that is closer to the heart than the distal end of the catheter, whereas if an artery is selected (blood flows away from the heart), downstream is a location on the limb that is farther from the heart than the distal end of the catheter. In FIG. 7, balloon catheter 212a is in branch artery 242, and thus, distal flow restricting cuff 222 is employed because proximal flow restricting cuff 220 (see FIG. 6) is closer to the heart than distal end of the catheter 212a, not farther from the heart, as is required when balloon catheter 212a is used in an artery.

The distance between balloon 244 and distal flow restricting cuff 222 (or proximal flow restricting cuff 220, if catheter 212a is disposed in a vein) determines the extent of the region that will be isolated from the patient's circulatory system (i.e., desired tissue location 218a). Balloon 244 and the flow restricting cuff are activated, isolating desired tissue location 218a from the patient's circulatory system. The desired medical agent is injected from distal end 224 of catheter 212a, and again, the practitioner waits for the specified desired period of time. After that time period has elapsed, the balloon and the distal flow restricting cuff are deactivated to reestablish the normal blood flow in the limb.

Again, if treatment is required for a time period longer than 5-10 minutes, the treatment will preferably be provided in a series of short intervals (e.g., each of 5 minutes or less) spread out over a longer period of time. Between each short treatment interval, the flow restricting cuff and balloon will be relaxed, enabling normal blood flow to resume. As before, for as long as continued treatment is required, it is preferable that the catheter remain in place until no further treatment is required.

FIG. 8 illustrates a different embodiment, in which a fluid displacement cuff 246 is applied between proximal flow restricting cuff 220 and distal flow restricting cuff 222 to first compress and then to remove a substantial amount of fluid from the blood and lymph vessels in the target region. The proximal and distal cuffs are then inflated to isolate desired tissue location 218, and fluid displacement cuff 246 is released. The therapeutic agent is then administered within desired tissue location 218. While FIG. 8 illustrates catheter 212 disposed in vein 214, it will be understood that catheter 212 can instead be introduced into artery branch 242, as shown in FIG. 7. The specific vessel in which catheter 212 is disposed is not critical, as long as the medical agent is released into a vessel adjacent to the target treatment site (in this case, stenosis 234).

After a suitable time has elapsed, sufficient for transfer of the medical agent across the walls of the capillaries and venules and into the surrounding tissues, fluid displacement cuff 246 is reinflated to displace any residual medical agent back into the administration catheter from the vessels within the desired tissue location. This step further reduces the amount of therapeutic agent that is released into other regions of the patient's body.

As described above, the distance between proximal flow restricting cuff 220 and distal flow restricting cuff 222 determines the extent of the region that is isolated from the patient's circulatory system (i.e., desired tissue location 218). In the embodiment illustrated in FIG. 8, it is again preferable that any treatment over a time period substantially longer than 5-10 minutes will be provided in a series of short intervals (such as 5 minutes or less) spread out over a longer period of time. Between each short treatment interval, fluid displacement cuff 246 is reinflated to displace any residual medical agent in the vessels at the desired tissue location back into the administration catheter. The proximal and distal flow restricting cuffs are then relaxed, enabling normal blood flow to resume. As before, for as long as continued treatment is required, it is preferable that the catheter remain in place until no further treatment is required.

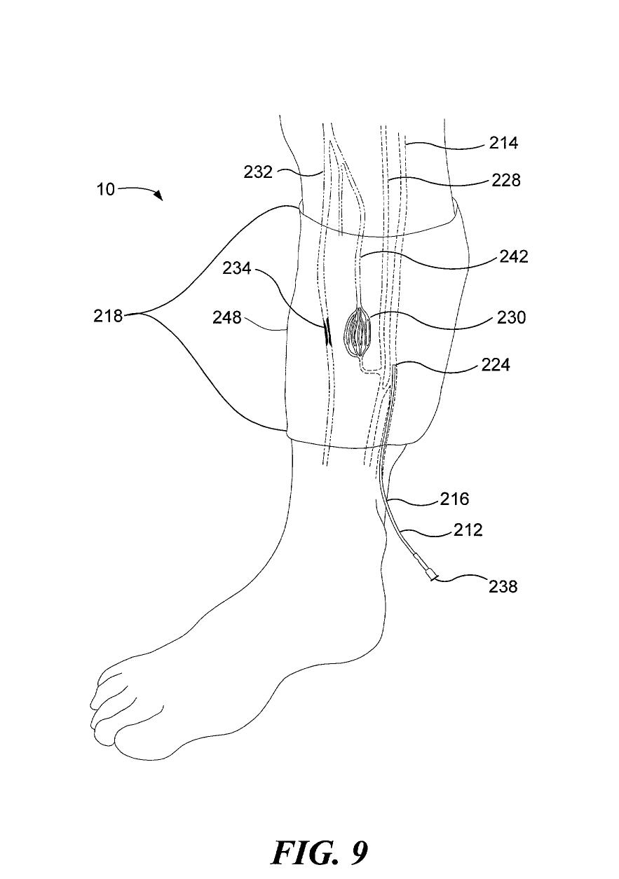

FIG. 9 illustrates an embodiment in which a single restricting cuff 248 is used to isolate desired tissue location 218 from the rest of the patient's circulatory system. Note that restricting cuff 248 is substantially larger in area (i.e., the area of the patient's limb that is compressed by the cuff) than either proximal flow restricting cuff 220 or distal flow restricting cuff 222. In fact, restricting cuff 248 is sized to substantially encompass the extent of desired tissue location 218. Restricting cuff 248 can comprise a conventional tourniquet, but more preferably, comprises a pressure cuff that can be manually or automatically inflated. As shown in FIG. 9, catheter 212 is inserted into vein 214 through puncture site 216. It should be understood that when restricting cuff 248 is employed, a catheter could be inserted into other veins or arteries as well. Note that if restricting cuff 248 is used, a balloon catheter, such as balloon catheter 212a in FIG. 7 is also required. Restricting cuff 248 removes a substantial amount of fluid from the blood and lymph vessels in the target region in the same fashion as fluid displacement cuff 246.

The general manner in which single restricting cuff 248 and catheter 212, as illustrated in FIG. 9, are employed is similar to the method previously described, except that only a single flow restricting cuff is activated/deactivated. Catheter 212 is first inserted into the vein or artery selected, and advanced to the desired location in the same manner. Single flow restricting cuff 248 is disposed such that the flow restricting cuff overlaps the distal end of the catheter and so that flow restricting cuff 248 is located substantially over the desired tissue location (note that the extent of single flow restricting cuff actually defines the region in which normal blood flow is occluded in the limb).

Flow restricting cuff 248 is activated, isolating desired tissue location 218 from the patient's circulatory system. The desired medical agent is injected from distal end 224 of catheter 212, and again the practitioner waits for the specified or desired period of time. After that time period has elapsed, flow restricting cuff 248 is deactivated to reestablish the normal blood flow. The time period that normal blood flow is occluded is minimized, and if treatment is required for a time period substantially longer than 5-10 minutes, the treatment will preferably be provided in a series of short intervals (such as 5 minutes or less) spread out over a longer period of time. Between each short treatment interval, the flow restricting cuff is relaxed, enabling normal blood flow to resume. It is preferable that the catheter remain in place until no further treatment is required.

With respect to any of the embodiments illustrated in FIGS. 6-9, the sequence of activating the fluid displacement cuff (the embodiment of FIG. 8 only), activating one or both of the proximal flow restricting cuff and the distal flow restricting cuff (or the single large flow restricting cuff of the embodiment in FIG. 9), de-activating fluid displacement cuff (the embodiment of FIG. 8 only), injecting the medical agent, waiting a specified desired period of time, reactivating the fluid displacement cuff (the embodiment FIG. 8 only) and then deactivating one or both of the proximal flow restricting cuff and the distal flow restricting cuff (or the single large flow restricting cuff of the embodiment in FIG. 9) to reestablish the blood flow can be performed manually. However, the procedure is preferably automated to provide a simpler and more reliable method of treatment. Therefore, the embodiments of FIGS. 6-9 are preferably automated.

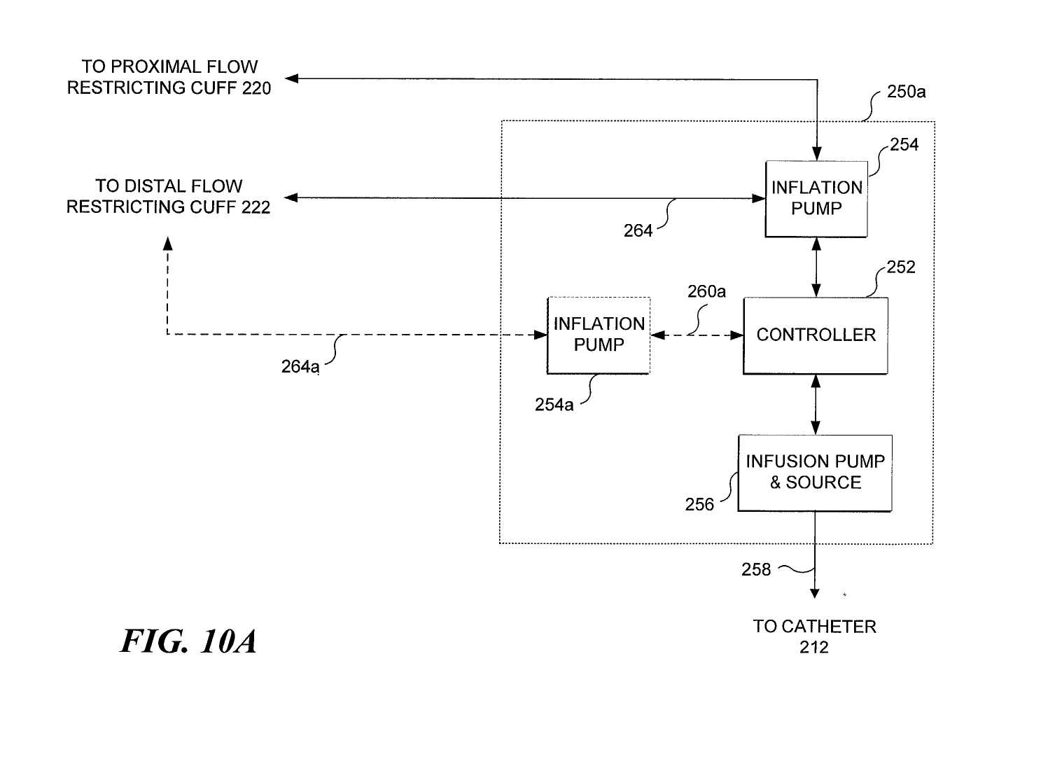

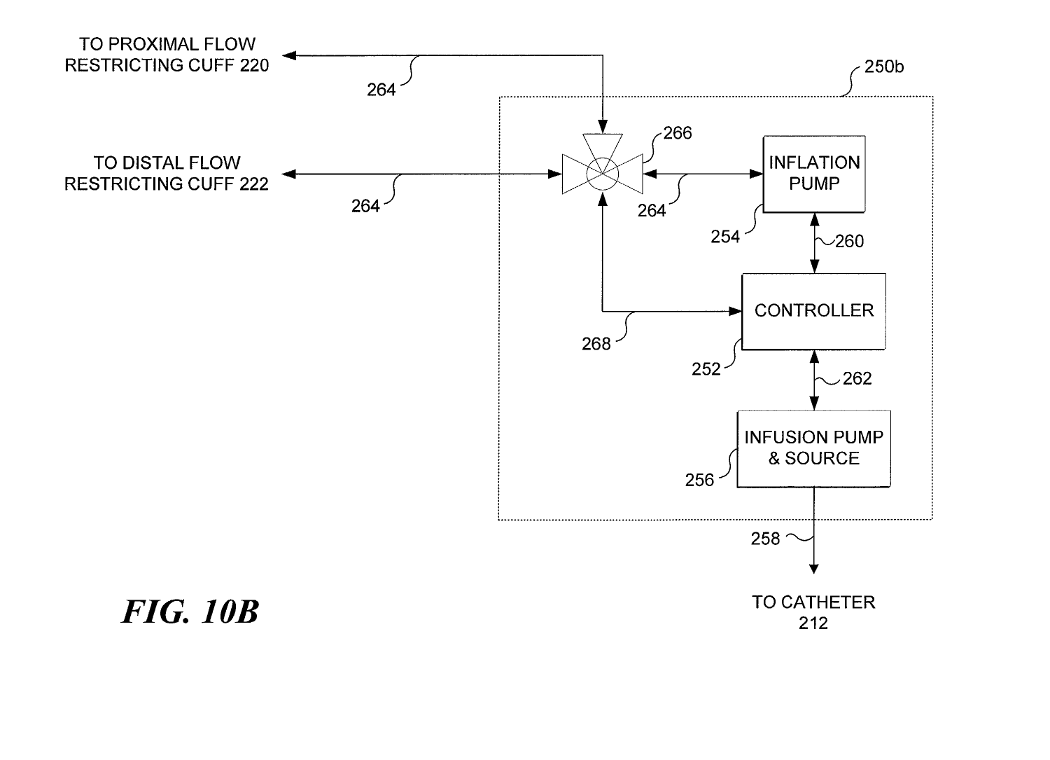

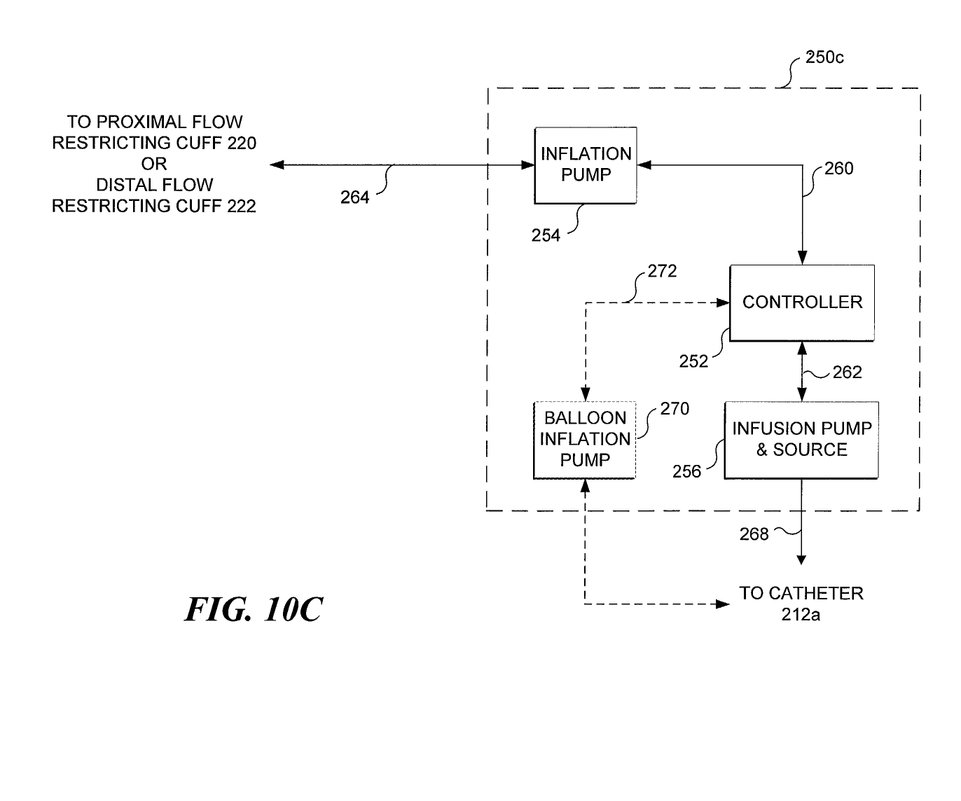

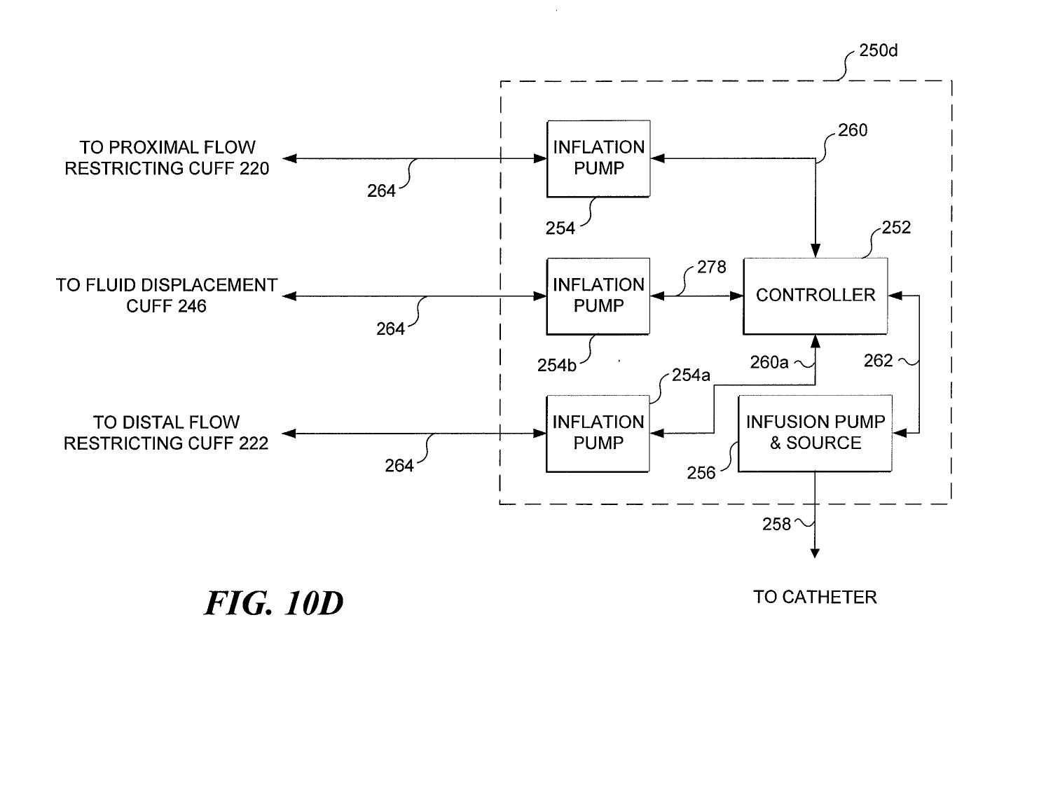

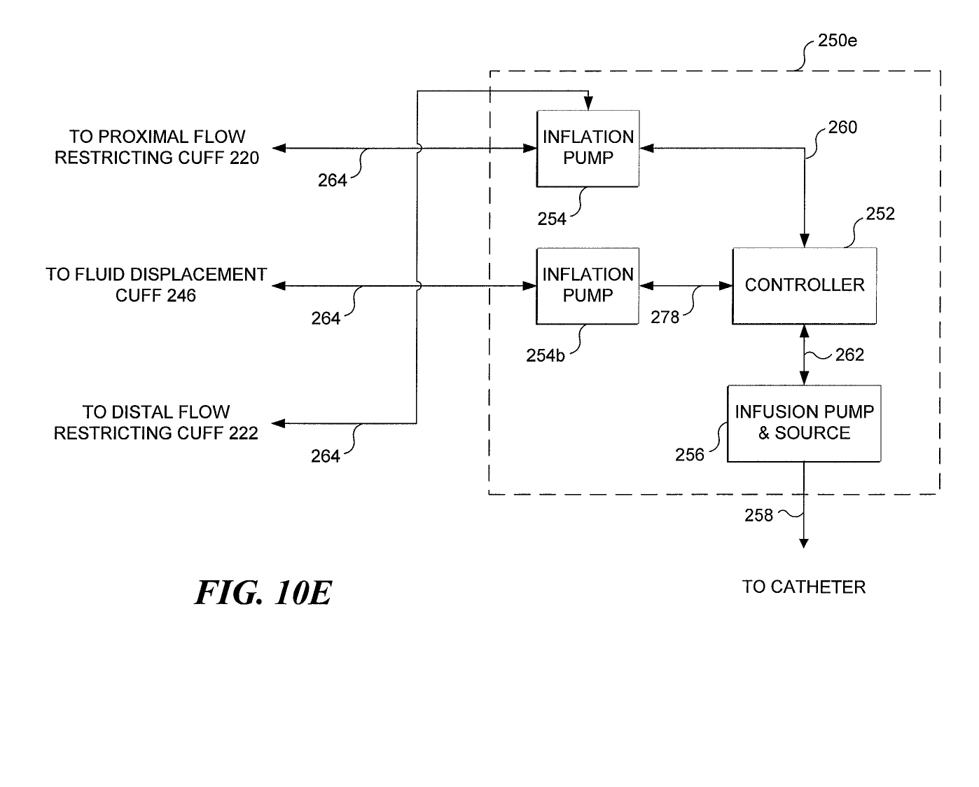

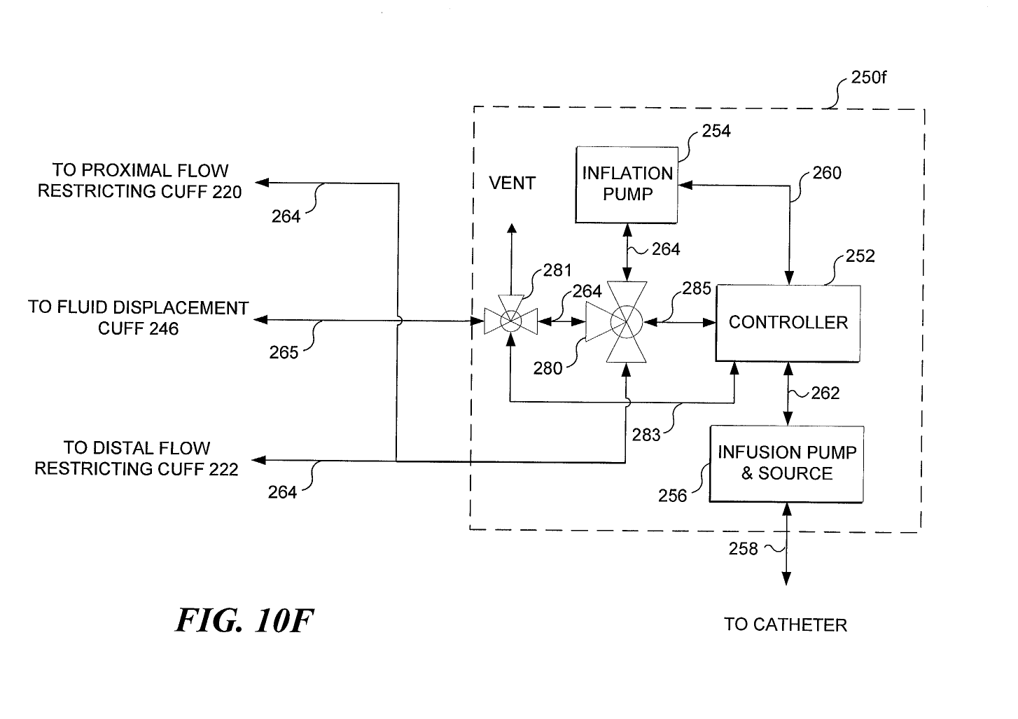

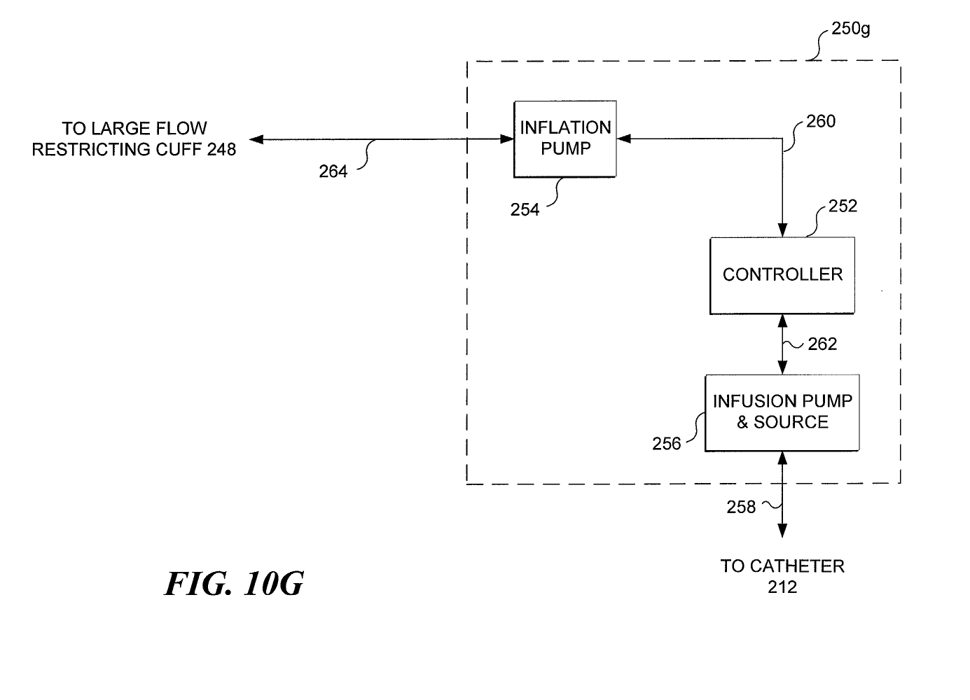

FIGS. 10A-10G illustrate automated systems 250a-250g, each of which automate one of the embodiments disclosed in FIGS. 6-9. In general, each automated system includes a controller that automatically controls blood flow into and out of the affected limb, and administers a medicinal agent to the treatment site in the limb at predetermined intervals of time. Also included is at least one inflation pump and an infusion pump, and an infusion fluid supply. It should be noted that for any of the embodiments illustrated in FIGS. 6-9 to be automated, the flow restricting and fluid displacement cuffs must be pressure cuffs coupled to an automatically controlled inflation pump, rather than tourniquets. Clearly, pressure cuffs are adaptable to automated control, whereas tourniquets require manual manipulation.

Each of the embodiments shown in FIGS. 10A-10G includes a controller 252. It should be noted that FIGS. 3 and 4, and the discussion relating to those figures provide detail on controllers that can be beneficially employed to control systems 250a-250g. Thus, controller 252 includes rest/pressure timer 70, pressure threshold comparator 76, dosage timer 80, dosage comparator 92, logic gate 82, and logic gate 86. The function and interaction of these elements have been described in detail above.

Processor-based controller 100, illustrated in FIG. 4 and discussed in detail above, can also be beneficially employed as controller 252 in automated systems 250a-250g. As noted above, controller 100 may be a specialized device designed specifically for the purpose of controlling delivery of the medicinal agent to a treatment site, or a general computing device, such as a personal computer that is programmed to do so. Controller 100 includes processor 102, memory 104 (RAM, ROM, and optionally, a permanent storage media), input interface 106, inflation interface 112, and infusion interface 118. While not integral to controller 100, keyboard 108 is used to enter commands and parameters employed to control delivery of a medicinal agent to a treatment site. Displays and switches 110 are additionally or alternatively used to enter commands and parameters to control delivery of the medicinal agent. It should further be noted that it is preferred for any inflation pumps employed in automated systems 250a-250g to a include pressure sensor. The use of such pressure sensors are described above in detail with respect to FIGS. 3 and 4.

Referring to FIG. 10A, an automated system 250a is adapted to provide automatic control for the embodiment illustrated in FIG. 6. A controller 252 enables automatic control of blood flow into and out of the desired target tissue, and administers a medicinal agent to the treatment site in the limb at predetermined intervals of time. While a conventional personal computer or other more general computing device (neither separately shown) can be used for controlling and automating the repetitive infusion of the medicinal agent through a catheter, and controlling the pressurization of pressure cuffs (such as proximal flow restricting cuff 220 and a distal flow restricting cuff 222), it is likely that controller 252 will be an application specific integrated circuit (ASIC) specifically designed for this purpose. The controller may be battery powered or powered from an internal or external ac line power supply (not shown), as generally described above. Controller 252 controls an infusion pump and a source 256 via signals conveyed over an infusion control line 258 and controls an inflation pump 254 via signals conveyed over an inflation control line 260.

Luer fitting 238 (see FIG. 6) on catheter 212 is coupled in fluid communication with infusion pump and source 256 through an infusion line 262. The infusion pump and source includes a small reservoir, vial, or other container (not separately shown) in which the medicinal agent is stored. When the medicinal agent is administered manually, a conventional syringe can be used to force the medicinal fluid through the catheter to the treatment site. In the automated embodiment shown in FIG. 10A, infusion pump 256 preferably comprises an automated syringe pump, a cassette pump, peristaltic pump, or other suitable medicinal fluid pump that is controlled in response to a signal received from controller 252 over infusion control line 258.

Inflation pump 254 is connected in fluid communication with proximal flow restricting cuff 220 via flexible tubes 264 and preferably comprises a standard pneumatic inflation pump of a size and volumetric rating suitable for pneumatically inflating proximal flow restricting cuff 220 to a pressure sufficient to substantially stop blood flow into and out of a limb of a patient, in response to a signal received from controller 252 over inflation control line 260. As shown, two separate flexible tubes 264 are coupled to inflation pump 254. While not shown, it should be understood that alternatively, a single flexible tube can be coupled to the inflation pump, branching into two lines to individually supply each restricting cuff with pressurized fluid.

An optional second inflation pump 254a is shown, since it may be desirable to inflate either proximal flow restricting cuff 220 or distal flow restricting cuff 222 at different times, rather that simultaneously. Under such circumstances, optional second inflation pump 254a is controllably connected to controller 252 via a control line 260a and is in fluid communication with distal flow restricting cuff 222 through flexible tube 264a.

An automated system 250b that enables individual activation of proximal flow restricting cuff 220 and distal flow restricting cuff 222 is illustrated in FIG. 10B, which shows a controllable valve 266 controllably connected to controller 252 via a control line 268. Controller 252 is thus enabled to selectively control valve 266 to determine the order that proximal flow restricting cuff 220 and distal flow restricting cuff 222 will be inflated and deflated.

Referring to both FIGS. 10A and 10B, once the system has been set up as shown, controller 252 activates inflation pump 254 (and inflation pump 254a, if required) to inflate proximal flow restricting cuff 220 and distal flow restricting cuff 222 to stop blood flow into and out of the desired tissue location 218 (see FIG. 6) for a specific (predefined desired) length of time. Once the appropriate inflation pressure is reached to stop blood flow in the limb, infusion of the medicinal agent commences with the delivery of the medicinal agent from infusion pump and source 256 through line 262 and catheter 212, into vein 214 through distal end 224. After a predetermined infusion time period has elapsed, inflation pump 254 (and inflation pump 254a, if used) releases the pressure in proximal flow restricting cuff 220 and distal flow restricting cuff 222, and blood flow is restored to desired tissue location 218. After a specified rest period, the inflation and infusion sequence is repeated. In this manner, small doses of medicinal agent are repetitively and safely infused into the treatment site.

FIG. 10C shows an automated system 250c adapted to provide automatic control for the embodiment illustrated in FIG. 7. Controller 252 enables automatic control of blood flow into and out of the desired target tissue, and administers a medicinal agent to the treatment site in the limb at predetermined intervals of time. Luer fitting 238 (see FIG. 7) on catheter 212a is coupled in fluid communication with infusion pump and source 256 through an infusion line 262. The infusion pump and source includes a small reservoir, vial, or other container (not separately shown) in which the medicinal agent is stored. As described above, infusion pump 256 preferably comprises an automated syringe pump, a cassette pump, peristaltic pump, or other suitable medicinal fluid pump that is controlled in response to a signal received from controller 252 over infusion control line 258.

Inflation pump 254 is connected in fluid communication with one of proximal flow restricting cuff 220 and distal flow restricting cuff 222 via flexible tubes 264. As discussed above, the use of a proximal or distal restricting cuff is a function of whether catheter 212a is inserted into a vein or artery. In either case, the restricting cuff must be located downstream (based on the normal flow of blood in the relevant type of vessel) from the distal end of catheter 212a. As before, inflation pump 254 preferably comprises a standard pneumatic inflation pump of a size and volumetric rating suitable for pneumatically inflating the flow restricting cuff to a pressure sufficient to substantially stop blood flow into and out of desired tissue location 218a, in response to a signal received from controller 252 over inflation control line 260.

Note also that an optional balloon inflation pump 270 is shown. As noted above, catheter 212a includes an inflatable balloon that occludes blood flow while the catheter is within a vein or artery, so that only a single external restriction cuff is required to define desired tissue location 218a. While it is contemplated that infusion pump 256 can also be used to inflate balloon 244 (see FIG. 7), it is anticipated that due to the very low volume required to inflate balloon 244, it will be preferable to provide a dedicated inflation pump to inflate balloon 244. In this embodiment, balloon inflation pump 270 is controllably connected to controller 252 via a control line 272 and is in fluid communication with balloon 244 via fluid line 274. While not shown, it should be understood that by incorporating a valve (such as valve 266 in FIG. 10B) balloon 244 can be inflated using inflation pump 254. Because the relative volumes and pressures required to inflate an external pressure cuff and a catheter balloon are so disparate, it is expected that a preferred embodiment will incorporate balloon inflation pump 270.

FIGS. 10D-10F illustrate automated systems 250d-250f, which are adapted to provide automatic control for the embodiment illustrated in FIG. 8. The three different embodiments relate to the use of one, two, or three different inflation pumps to control the proximal and distal restriction cuffs, and the fluid displacement cuff, respectively. In each embodiment, controller 252 enables automatic control of blood flow into and out of desired target tissue location 218 and administers a medicinal agent to the treatment site in the limb at predetermined intervals of time. Controller 252, the inflation pumps, and the infusion pumps are as described above.

Referring to FIG. 10D, in an automated system 250d, proximal flow restricting cuff 220, distal flow restricting cuff 222, and fluid displacement cuff 246 each are controlled by separate inflation pumps. Inflation pump 254 is coupled in fluid communication with proximal flow restricting cuff 220 via a flexible tube 264, while an inflation pump 254a is coupled in fluid communication with distal flow restricting cuff 222 via a flexible tube 264. The inflation pumps are controllably connected to controller 252 by inflation control lines 260 and 260a. An inflation pump 254b is in fluid communication with fluid displacement cuff 246 and is similarly controllably connected to controller 252 by an inflation control line 278.

The logic implemented by controller 252 first activates inflation pump 254b to compress and remove a substantial amount of fluid from the blood and lymph vessels in the target region within which the catheter is properly positioned. The proximal and distal cuffs are then inflated to isolate desired tissue location 218, and the pressure in fluid displacement cuff 246 is released. The medicinal agent is then administered within the desired tissue location when controller 252 activates infusion pump and source 256 with a signal provided over control line 262.

After a sufficient time has elapsed for transfer of the medical agent across the walls of the capillaries and venules and into the surrounding tissues, controller 252 activates inflation pump 254b to cause fluid displacement cuff 246 to be reinflated, thereby displacing any residual medical agent back into the administration catheter from the vessels disposed within the desired tissue location. This step reduces the amount of therapeutic agent that is released into other regions of the patient's body. Controller 252 then activates inflation pumps 254 and 254a to cause the proximal and distal flow restricting cuffs to relax (deflate), thereby reestablishing normal blood flow. For repeated treatment, after a predefined rest period has elapsed, the above steps are repeated.

In an automated system 250e shown in FIG. 10E, proximal flow restricting cuff 220 and distal flow restricting cuff 222 are inflated using inflation pump 254, while fluid displacement cuff 246 is inflated by separate inflation pump 254b. Inflation pump 254 is coupled in fluid communication with proximal flow restricting cuff 220 and distal flow restricting cuff 222 via flexible tubes 264. As noted above, either two separate flexible tubes can be employed (as shown), or a single flexible tube can optionally be provided with a T-connector (not shown) that branches into two separate tubes, each coupled to a different one of the flow restricting cuffs. As before, inflation pump 254 is controllably connected to controller 252 via inflation control line 260. Inflation pump 254b is coupled in fluid communication with fluid displacement cuff 246 via flexible tube 276 and is controllably connected to controller 252 via inflation control line 278.

The logic controlling the controller 252 in the embodiment shown in FIG. 10E first activates inflation pump 254b to compress and remove a substantial amount of fluid from the blood and lymph vessels in the target region, then activates inflation pump 254 to inflate the proximal and distal cuffs to isolate desired tissue location 218, and releases fluid displacement cuff 246. The therapeutic or medicinal agent is then administered within the desired tissue location when controller 252 activates infusion pump and source 262. After the required time has elapsed, controller 252 activates inflation pump 254b to cause fluid displacement cuff 246 to be reinflated, thereby displacing any residual medical agent back into the administration catheter from the vessels within the desired tissue location. Controller 252 next activates inflation pump 254 to cause the proximal and distal flow restricting cuffs to deflate or relax, thereby reestablishing normal blood flow. For repeated treatment, after a predefined rest period has elapsed, the above steps are repeated.

Yet another embodiment for automating the system in FIG. 8 employs only a single inflation pump, and uses a valve to selectively determine which of the two restriction cuffs and the displacement cuff is inflated by the pump. FIG. 10F shows such an automated system 250f, in which proximal flow restricting cuff 220, distal flow restricting cuff 222, and fluid displacement cuff 246 are each inflated using a single inflation pump. Inflation pump 254 is coupled in fluid communication with a valve 280 via flexible tube 264. Valve 280 is controllably connected to control 252 via valve control line 285. Valve 280 is selectively placed in fluid communication with any one of proximal flow restricting cuff 220, distal flow restricting cuff 222, and a bleed valve 281 via flexible tubes 264. Note also that a bleed valve 281 is disposed between valve 280 and fluid displacement cuff 246. Bleed valve 281 is controllably connected to controller 252 via a control line 283, and one outlet of the bleed valve is coupled in fluid communication with fluid displacement cuff 246 through a flexible tube 265. The purpose of bleed valve 281 is to enable fluid displacement cuff 246 to be deflated without requiring valve 280 to be placed in fluid communication with fluid displacement cuff 246 and deactivating inflation pump 254. Thus, inflation pump 254 can be used to actively inflate proximal flow restricting cuff 220 and distal flow restricting cuff 222, even while fluid displacement cuff 246 is deflated.

The following steps occur after the catheter is properly positioned in a vessel. The logic controlling controller 252 first activates valve 280, using valve control line 285, to place inflation pump 254 in fluid communication with fluid displacement cuff 246. Controller 252 will next activate inflation pump 254 to compress and remove a substantial amount of fluid from the blood and lymph vessels in the target region (by activating fluid displacement cuff 246). Next, the logic controlling controller 252 will again activate valve 280, using valve control line 285, to place inflation pump 254 in fluid communication with either proximal flow restricting cuff 220 or distal flow restricting cuff 222. Controller 252 will then activate inflation pump 254 to activate the selected restriction cuff, and the process of changing the valve position and activating the inflation pump will be repeated for the other restriction cuff, thereby isolating desired tissue location 218 (shown in FIG. 8). Alternatively, valve 280 can place inflation pump 254 in fluid communication with both proximal flow restricting cuff 220 and distal flow restricting cuff 222, so that each cuff is inflated at the same time. Once all cuffs are inflated, the logic actuates bleed valve 281 to relax or deflate fluid displacement cuff 246. The therapeutic agent is then administered within the desired tissue location as controller 252 activates infusion pump and source 262.

As described above, after a suitable time has elapsed, controller 252 closes bleed valve 281, and activates valve 280 so that fluid displacement cuff 246 is in fluid communication with inflation pump 254, thereby causing fluid displacement cuff 246 to be reinflated. The inflation of the fluid displacement cuff forces any residual medical agent back into the administration catheter. Next, the logic controlling controller 252 again activates valve 280, using valve control line 285, to place inflation pump 254 in fluid communication with either proximal flow restricting cuff 220 or distal flow restricting cuff 222. Controller 252 then deactivates inflation pump 254 to deflate the selected restriction cuff, and the other of proximal flow restricting cuff 220 or distal flow restricting cuff 222 is placed in fluid communication with the deactivated inflation pump, causing the other cuff to similarly be deflated. As noted above, valve 280 can place inflation pump 254 in fluid communication with both proximal flow restricting cuff 220 and distal flow restricting cuff 222, so that each cuff is deflated at the same time. Deflating the proximal and distal flow restricting cuffs reestablishes normal blood flow in the patient's limb. For repeated treatment, after a predefined rest period has elapsed, the above steps are repeated.

As noted above, while not shown, it is contemplated that system 250f could incorporate a two-way valve, rather than three-way valve 280. In such an embodiment, in a first position, a two-way valve would be in fluid communication with fluid displacement cuff 246. In a second position, the two-way valve would simultaneously be in fluid communication with both proximal flow restricting cuff 220 and distal flow restricting cuff 222. The control sequence disclosed above would be modified accordingly.

Referring now to FIG. 10G, an automated system 250g is adapted to automatically control the embodiment shown in FIG. 9, in which a single, relatively large (as compared to proximal flow restricting cuff 220 and distal flow restricting cuff 222) flow restricting cuff 248 is used to isolate desired tissue location 218 from the rest of a patient's circulatory system. Automated system 250g includes inflation pump 254, which is coupled in fluid communication with large flow restricting cuff 248 via flexible tube 264. Inflation pump 254 is controllably connected to controller 252 by inflation control line 260. Controller 252 is also controllably connected to infusion pump and source 256 by control line 262.

Once catheter 212 has been properly positioned and automated system 250g has been initialized, the logic implemented by controller 252 activates inflation pump 254 to inflate large flow restricting cuff 248, thereby isolating desired tissue location 218 from blood flow through the limb on which the cuff is applied. The therapeutic agent is then administered within the desired tissue location as controller 252 activates infusion pump and source 256. After a required time has elapsed, controller 252 activates inflation pump 254, causing large flow restricting cuff 248 to be deflated, thereby reestablishing normal blood flow. For repeated treatment, after a predefined rest period has elapsed, the above steps are repeated.

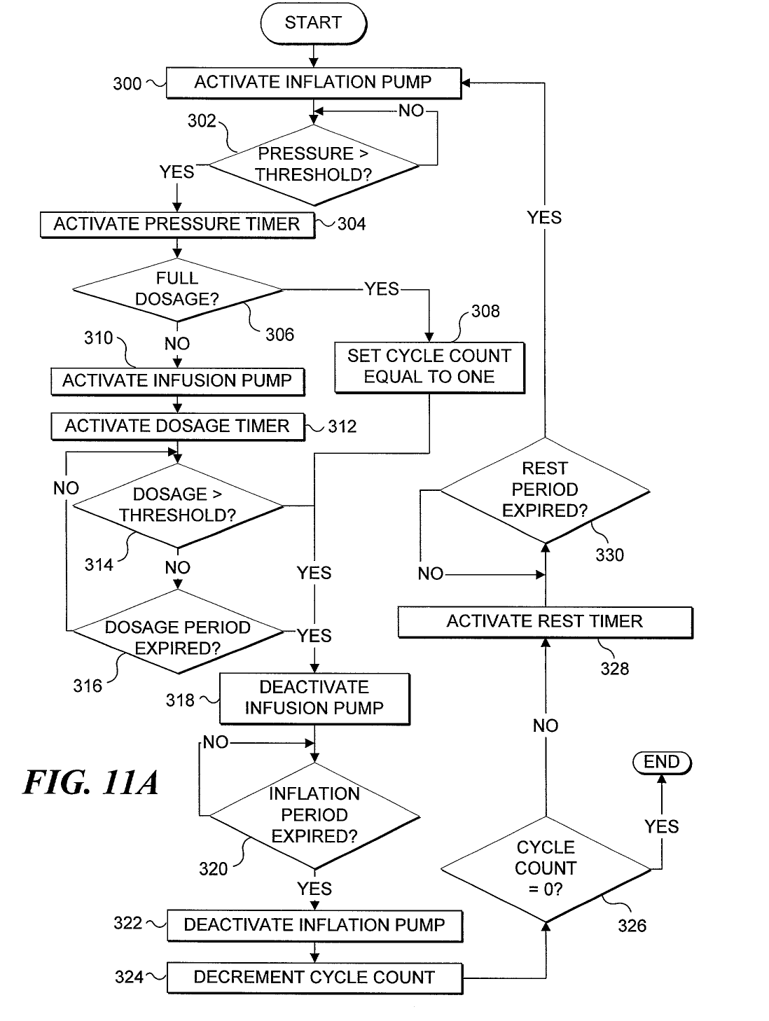

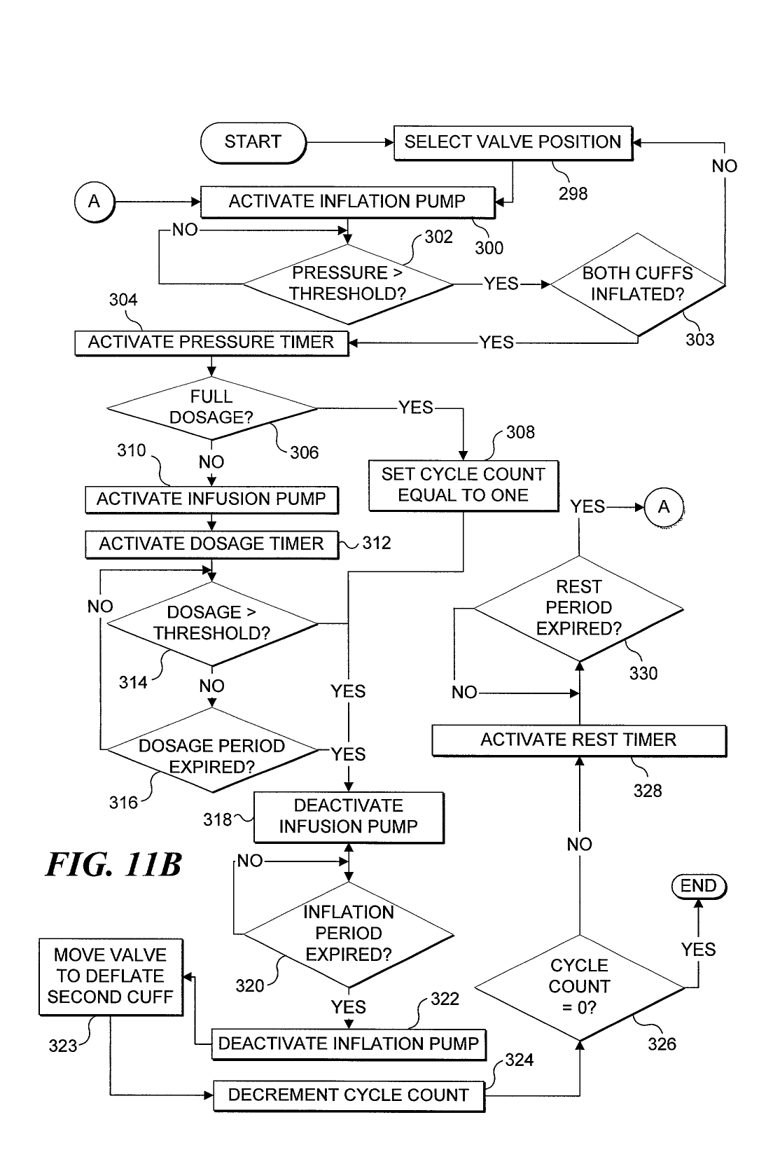

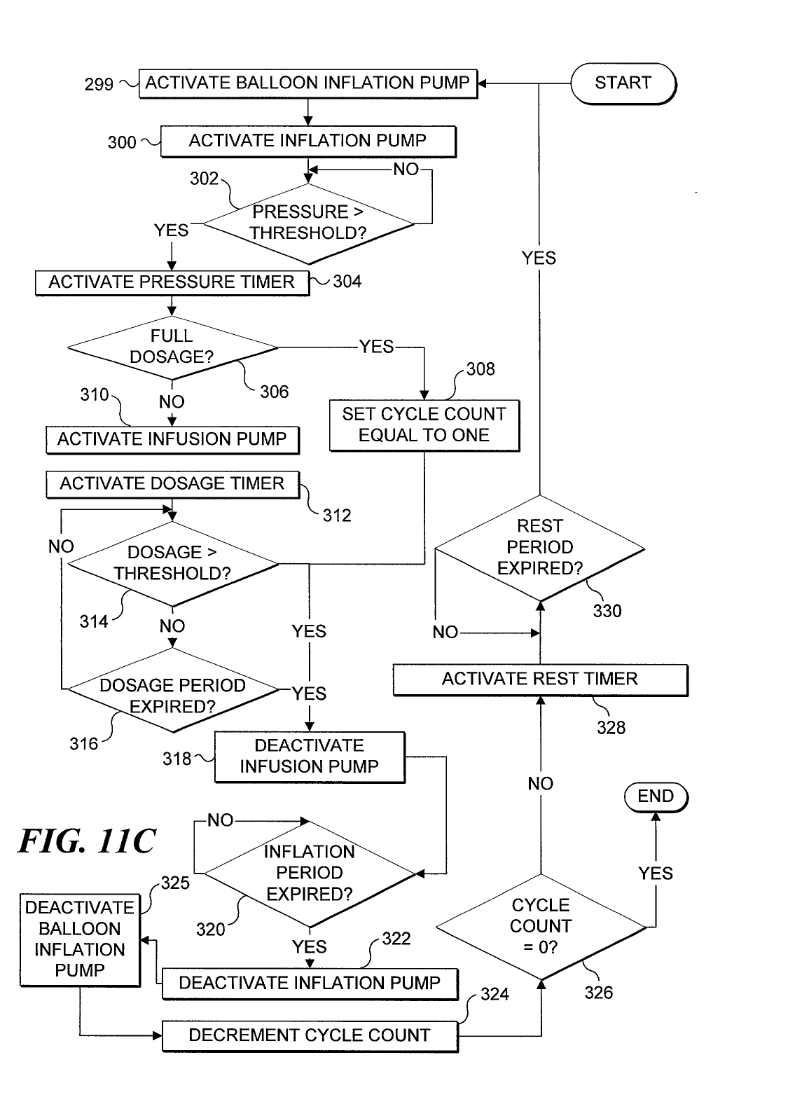

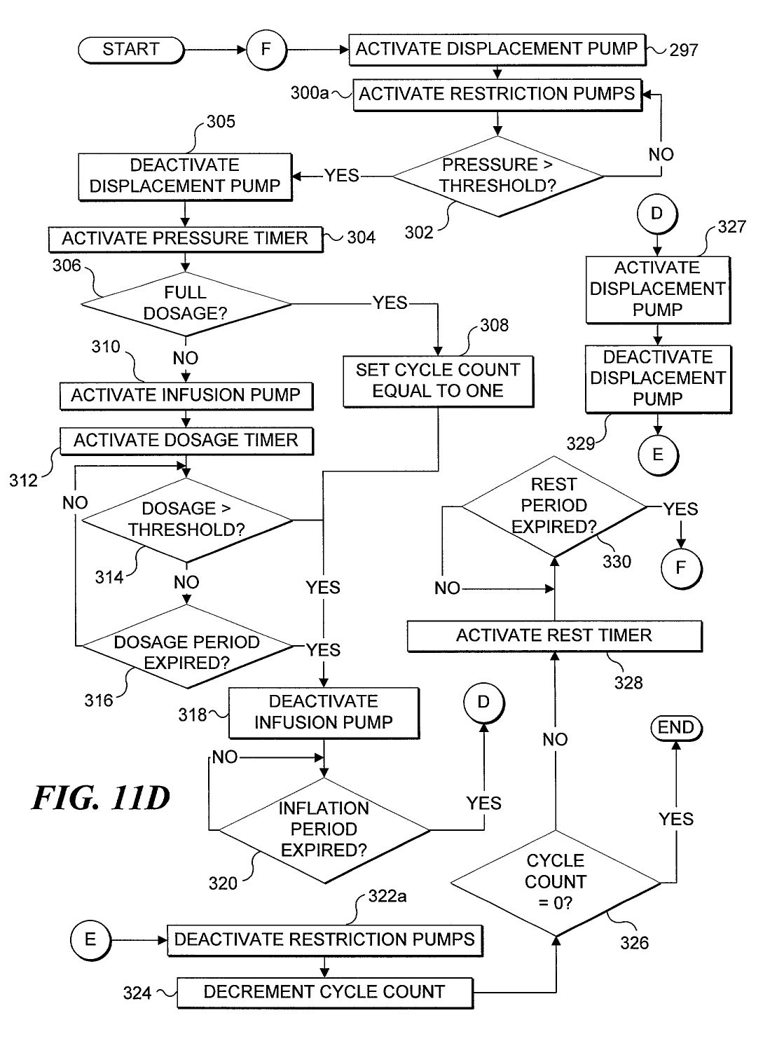

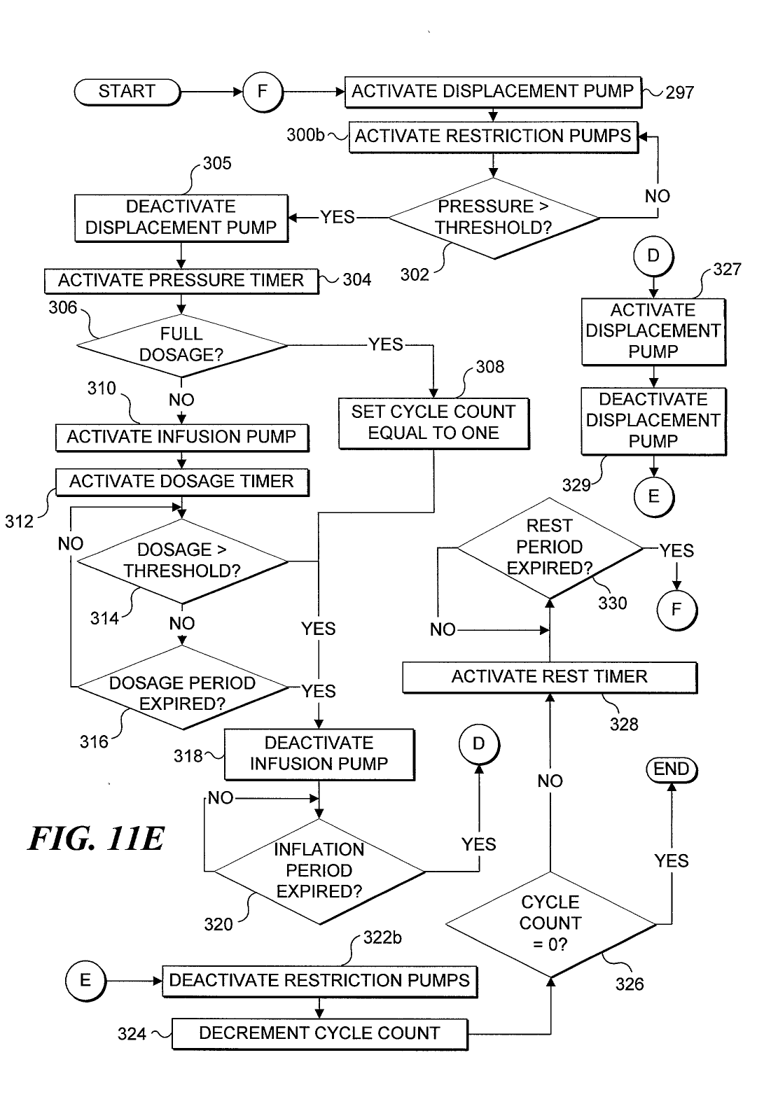

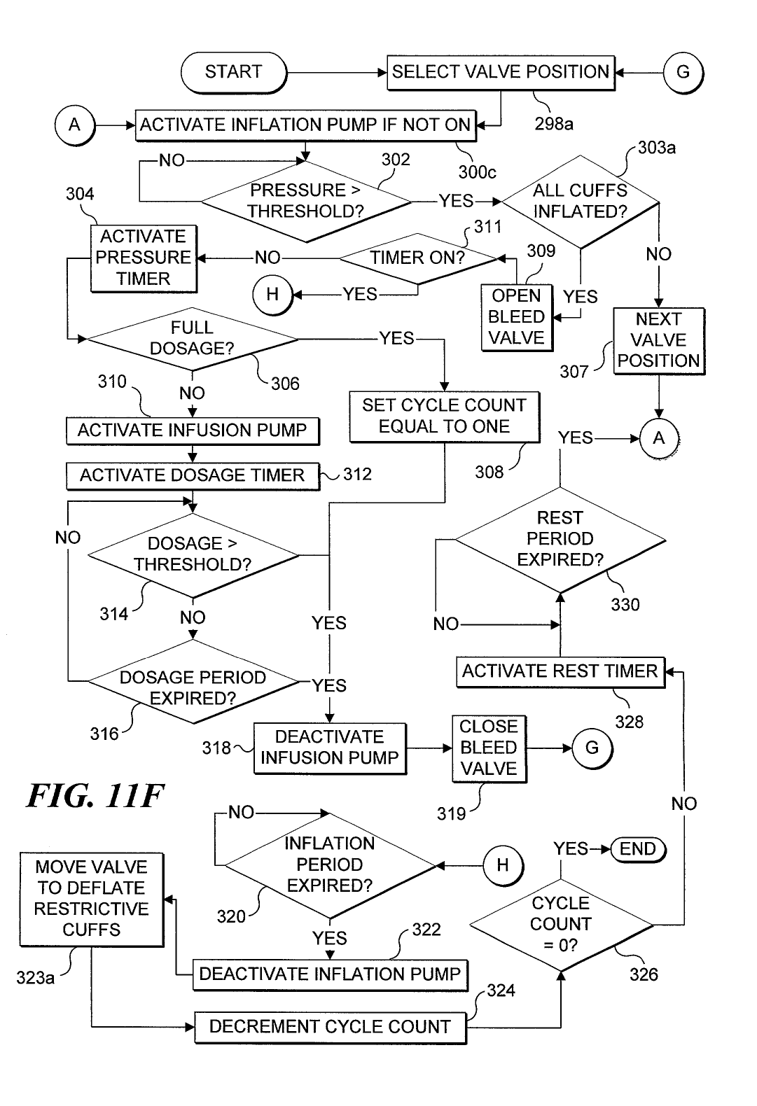

While the above descriptions relative to automated systems 250a-250g have include general steps employed for controlling the respective embodiments, the following discussion regarding FIGS. 11A-11F provides a more detailed description of the control logic that is implemented in software or in hardwired logic to control repetitive cycles of infusion of a medicinal agent to a treatment site in a limb of a patient. It should be noted that the logic illustrated in the following figures is very similar to the logical process illustrated in FIG. 5; however, that embodiment employs only a single restrictive cuff and inflation pump, while some of the automated systems 250a-250g include additional restrictive cuffs, displacement cuffs, valves, and/or inflation pumps.

FIG. 11A illustrates the control logic used in conjunction with automated system 250a of FIG. 10A, wherein a single inflation pump controls both the proximal and distal flow restrictive cuffs. At a block 300, inflation pump 254 is activated to pressurize proximal flow restrictive cuff 220 and distal flow restrictive cuff 222 to stop the flow of blood in desired tissue location 218. It should be noted that as illustrated, the logic simultaneously fills both proximal flow restrictive cuff 220 and distal flow restrictive cuff 222. Both cuffs will generally be quite similar, and should require substantially the same pressure for proper inflation. Accordingly, a single pressure sensor in inflation pump 254, in fluid communication with flexible tubes 264 (to each of proximal flow restrictive cuff 220 and distal flow restrictive cuff 222) is sufficient to provide controller 252 with a signal indicative of proper inflation of both proximal flow restrictive cuff 220 and distal flow restrictive cuff 222. If there is a concern that different pressures might be required for each cuff, the logic could be modified to enable one proximal flow restrictive cuff 220 and distal flow restrictive cuff 222 to be inflated first, and then the other of proximal flow restrictive cuff 220 and distal flow restrictive cuff 222 to be inflated next. For such step-wise inflation, a two-way valve must be connected into the flexible tube servicing the cuffs and must be of a type that ensures the first cuff inflated remains inflated when inflation pump 254 is employed to inflate the other cuff.

Returning to FIG. 11A, at a decision block 302, the pneumatic pressure in the flow restrictive cuffs is compared with a predetermined pressure threshold value sufficient to stop blood flow in the limb through desired tissue location 218. The logic loops until the detected pressure value in the flow restrictive cuffs is greater than the predetermined pressure threshold value. A pressure timer is then activated at a block 304 to begin a predetermined inflation period during which the flow of blood in desired tissue location 218 is stopped.

A decision block 306 determines if a predetermined total dosage of the medicinal agent has been administered. The amount of medicinal agent administered is determined by a flow transducer in the infusion pump or other sensor and compared with a predetermined total dosage value that can be set by a medical practitioner as a desired dosage or as a maximum allowable dosage. If a full total dosage of the drug has already been administered so that no further drug infusion is required by the infusion pump, then the cycle count is set to one in a block 308 and the infusion pump is deactivated at a block 318. The logic can be modified so that a full dosage result will also deactivate the inflation pump, as indicated in a block 322, causing blood flow in desired tissue location 218 to be immediately enabled. However, the logic shown maintains the pressure in the flow restrictive cuff for the entire inflation period so that any manually administered drug may be perfused into the tissue of the treatment site without blood flow carrying away the drug.

If a full total dosage has not yet been administered, the infusion pump is activated in a block 310, and a dosage timer is activated in a block 312. A decision block 314 determines whether the quantity of the medicinal agent delivered during a current cycle is greater than a desired dosage threshold. If so, then the infusion pump is deactivated in block 318. If the dosage threshold for the current cycle has not been reached, a decision block 316 determines whether the dosage period established by the dosage timer has expired. If the dosage period has not yet expired, the dosage of the drug delivered is checked again at decision block 314. When the dosage period has expired or the dosage is greater than the predetermined threshold, the infusion pump is deactivated at block 318.

A decision block 320 then determines whether a current inflation period has expired, as determined by the pressure timer. Until the inflation period has elapsed, the logic loops, providing time for the delivered drug to perfuse the tissue of the treatment site while the blood flow is stopped. Once the inflation period has expired, the inflation pump is deactivated at step 322. Because block 322 concludes a cycle of medicinal agent infusion, a cycle counter is decremented in a block 324. A decision block 326 then determines whether all of a predetermined number of cycles of infusion of the medicinal agent have been completed. If all of the infusion cycles have been completed, the process ends.

If additional infusion cycles remain, the rest timer is activated in a block 328. A decision block 330 then determines whether the rest period has expired, as determined by the rest timer. Until the rest period has elapsed, the logic loops, providing time for blood to resume flowing in the limb and treatment site, reoxygenating tissue in the portion of the limb where blood flow was prevented. Once the rest period has expired, the next infusion cycle begins by reactivating the inflation pump at block 300.

With respect to FIG. 10A, it has been contemplated (as described above), that optional inflation pump 254a can be beneficially incorporated into automated system 250a. in such an embodiment (the logic illustrated in FIG. 11A), the pressure timer in block 304 would not be activated until each inflation pump indicates that the pressure threshold had been reached (blocks 300-302).

FIG. 11B shows a logical process which has been specifically adapted to control automated system 250b of FIG. 10B and is quite similar to that illustrated in FIG. 11A. The changes required include the incorporation of blocks 298, 303, and 323. The balance of the logic is the same as described above. The first change is the incorporation of block 298, in which the logic selects a valve position, such that inflation pump 254 is in fluid communication with one of proximal flow restrictive cuff 220 and distal flow restrictive cuff 222. The logic then moves to block 300, which as described above requires the logic to activate inflation pump 254. In block 302, once the logic determines the pressure threshold is met, the logic now moves to decision block 303 and determines if both proximal flow restrictive cuff 220 and distal flow restrictive cuff 222 are inflated. If either cuff is not inflated, the logic loops back to block 298, and the valve is moved to select the other of proximal flow restrictive cuff 220 and distal flow restrictive cuff 222. If in decision block 303 the logic determines that both cuffs are inflated, the logic proceeds to block 304, and the steps as described above are similarly executed until block 322, in which the inflation pump is deactivated. Deactivating the pump will cause the cuff that is in fluid communication with the pump to be deflated. Note that only one cuff is in fluid communication with the pump, and that the other cuff will remain inflated (as it is isolated from inflation pump 254 by valve 266).

From block 322, the logic now moves to new block 323, and the logic changes the position of the valve, to place the other cuff in fluid communication with the deactivated inflation pump, thereby causing the other cuff to deflate. The logic then continues as described above, until in block 302 the logic moves once again to block 303 to determine if both cuffs are inflated. Note that while FIG. 11B shows block 330 leading to connector A, as opposed to block 300 in FIG. 11A, connector A leads to block 300, so the result is the same.

It should also be noted that the incorporation of a valve in automated system 250b makes it possible to modify the logic controlling the system to deactivate inflation pump more rapidly, as long as valve 266 is capable of simultaneously isolating proximal flow restrictive cuff 220 and distal flow restrictive cuff 222 from inflation pump 254. In such an embodiment (logic not shown), once both cuffs are inflated, valve 266 is manipulated to isolate both proximal flow restrictive cuff 220 and distal flow restrictive cuff 222 from inflation pump 254, so that the pump can be deactivated without deflating the cuffs. Then, instead of deactivating the inflation pump in block 322, valve 266 is manipulated to place both proximal flow restrictive cuff 220 and distal flow restrictive cuff 222 in fluid communication with the atmosphere, so that the pressure in the cuffs is released, thereby deflating the cuffs.

FIG. 11C shows a logical process that is also quite similar to that illustrated in FIG. 11A, and which has been specifically adapted to control automated system 250c of FIG. 10C. The changes required include the incorporation of blocks 299 and 323. The balance of the logic is as described above. The first change is the incorporation of block 299, in which the logic activates balloon inflation pump 270 to inflate balloon 244 of catheter 212a (see FIG. 7). The logic then activates inflation pump 254 in block 300. Desired tissue location 218a (see FIG. 7) is defined by balloon 244 and one external cuff (either proximal flow restrictive cuff 220 or distal flow restrictive cuff 222, depending on whether catheter 212a is disposed in a vein or artery, as discussed in detail above). Note that for automatic system 250c, blocks 300 and 302 are executed only for one of proximal flow restrictive cuff 220 and distal flow restrictive cuff 222. It should be noted that a pressure sensor could be employed in inflation pump 270 to ensure that balloon 244 is inflated sufficiently.

From block 304, the logic proceeds as described above until block 322, from which point the logic now continues with block 325 in which the balloon inflation pump is deactivated to deflate the catheter balloon. The logic then returns to block 324 and the cycle count is decremented as described above. The final change to the logical process described relative to that of FIG. 11A is that in FIG. 11C, the logic proceeds to block 299 from block 330, to activate both the balloon and cuff inflation pumps, as opposed to proceeding to block 300 to activate only the inflation pump.

The logical process illustrated in FIG. 11D shows how the logic described above has been adapted to control automated system 250d (see FIG. 10D). Automated system 250d includes three different inflation pumps, one for proximal flow restrictive cuff 220, one for distal flow restrictive cuff 222, and one for fluid displacement cuff 246. The changes to the logic include the incorporation of blocks 297, 305, 327 and 329, and connectors D, E, and F. Note that blocks 300 and 322 have been changed to blocks 300a and 322a, respectively, to account for activation and deactivation of both restriction cuff inflation pumps. The balance of the logic is as described above in connection with FIG. 11A.

The first change is the incorporation of block 297, in which the logic first activates fluid displacement cuff inflation pump 254b (see FIG. 8) to activate fluid displacement cuff 246, thereby compressing the tissue in the target area and removing a substantial amount of fluid from the blood and lymph vessels in desired tissue location 218. The logic then proceeds to block 300a, which varies from the logic described above only in that two flow restriction cuff inflation pumps are activated in block 300a, whereas the logic described in regard to FIG. 11A indicates that block 300 activates only a single inflation pump. Thus, in block 302, the logic will not proceed to new block 305 unless both pressure sensors (one for flow restriction cuff inflation pump 254, and one for flow restriction cuff inflation pump 254a) indicate that the proper pressure level has been obtained, indicating that both proximal flow restrictive cuff 220 and distal flow restrictive cuff 222 have been properly inflated.

Once the proper pressure conditions are established, the logic moves to block 305 and the fluid displacement cuff inflation pump is deactivated. If fluid displacement cuff 246 remains inflated, desired tissue location 218 remains compressed, and it would be difficult to introduce a medicinal agent into desired tissue location 218. Once the fluid displacement cuff inflation pump is deactivated, the logic proceeds to block 304, and the control logic is the same as described above for FIG. 11A until block 320, at which point the logic now proceeds (via connector D) to block 327. At this point, fluid displacement cuff inflation pump 254b is activated to cause fluid displacement cuff 246 to be reinflated, thereby displacing any residual medical agent back into the administration catheter from the vessels within the desired tissue location. The logic then advances to block 329, where fluid displacement cuff inflation pump 254b is deactivated, thereby causing fluid displacement cuff 246 to be relaxed or deflated. The logic then proceeds to block 322a (via connector E) and both inflation pumps 254 and 254a are deactivated, causing both the proximal and distal flow restricting cuffs to be deflated, thereby reestablishing normal blood flow. Note that block 322a differs from block 322 described above in FIG. 11A in that block 322 deactivates only a single inflation pump, whereas block 322a provides for deactivating two inflation pumps (one for the proximal flow restricting cuff, and one for the distal flow restricting cuff). The logic then proceeds to block 324, and the cycle count is decremented, as described above. The final change to the logical process described with respect to FIG. 11A is that in FIG. 11D, the logic advances to block 297 from block 330, to activate fluid displacement cuff inflation pump 254b, as opposed to proceeding to block 300, as described in connection with FIG. 11A.

FIG. 11E illustrates how the logic described above has been adapted to control automated system 250e (see FIG. 10E). Automated system 250e includes two different inflation pumps, one serving both proximal flow restrictive cuff 220 and distal flow restrictive cuff 222, and one that is used for fluid displacement cuff 246. The logic controlling automated system 250e is very similar to that described above with respect to FIG. 11D. The only differences are that blocks 300a and 322a have been changed to 300b and 322b, respectively, to account for the activation and deactivation of only a single restriction cuff inflation pump. Thus, in block 302, the logic examines data from only a single pressure sensor to determine if both proximal flow restrictive cuff 220 and distal flow restrictive cuff 222 have been properly inflated. It should be noted that blocks 300b and 322b are functionally identical to blocks 300 and 322 of FIGS. 11A-11C. Different numbers and descriptive text have been used in FIG. 11E to clearly distinguish the inflation pump that activates proximal flow restrictive cuff 220 and distal flow restrictive cuff 222 from the inflation pump that activates fluid displacement cuff 246. The balance of the logic illustrated in FIG. 11E is identical to that described with respect to FIG. 11D.

FIG. 11F illustrates the logic employed to control automated system 250f (see FIG. 10F), which is an automated embodiment of the treatment process shown in FIG. 8. Briefly, that embodiment employs fluid displacement cuff 246, spaced between proximal flow restricting cuff 220 and distal flow restricting cuff 222, to first compress and remove a substantial amount of fluid from the blood and lymph vessels in the target region (after the catheter is properly positioned). The proximal and distal cuffs are then inflated to isolate desired tissue location 218 and fluid displacement cuff 246 is released. The therapeutic agent is administered within the desired tissue location. Next, fluid displacement cuff 246 is once again inflated, to draw any residual therapeutic agent within the desired tissue location back into the catheter. Finally, the proximal and distal cuffs are released, and the automated system then waits until the next therapeutic infusion cycle begins.

While the logic controlling automated system 250f (which employs one inflation pump and a series of valves to control proximal flow restrictive cuff 220, distal flow restrictive cuff 222, and fluid displacement cuff 246) includes many elements described above, more changes have been required to enable automated system 250f to function than have been required for automated systems 250b-250e. The changes include the incorporation of blocks 298a, 303a, 305, 307, 309, 319 and 323a, as well as connectors A, G and H.

Once the logical control process is initiated in the start block, in block 298a the logic selects a valve position, such that inflation pump 254 is in fluid communication with fluid displacement cuff 246. Note that in the logical sequence of FIG. 11B, it was not critical which cuff was selected in block 298. However, in the logical sequence of FIG. 11F, inflation pump 254 must be placed in fluid communication with displacement cuff 246 in block 298a, as opposed to being placed in fluid communication with either or both of proximal flow restrictive cuff 220 and distal flow restrictive cuff 222.

The logic then moves to block 300,c and if the inflation pump is not already activated, it is activated, which is a slight change from the block 300 as described above, in which the logic merely activates the inflation pump. The change is required because the logic (via connectors A and G) can loop back to block 300c from locations in the logical sequence in which the inflation pump is already on. In block 302, once the logic determines if the pressure threshold is met (as described above), the logic moves to decision block 303a to determine if all cuffs (proximal flow restrictive cuff 220, distal flow restrictive cuff 222 and fluid displacement cuff 246) are inflated. Note that at start up, fluid displacement cuff 246 will be the only cuff inflated, and thus the logic moves to block 307, and valve 280 (FIG. 10F) is manipulated to place proximal flow restrictive cuff 220 and distal flow restrictive cuff 222 in fluid communication with inflation pump 254, so that proximal flow restrictive cuff 220 and distal flow restrictive cuff 222 are inflated (the logic moves via connector A to block 300c, and from block 302, returns to block 303a).

If in block 303a the logic determines that all cuffs are inflated, the logic moves to a block 309 and opens bleed valve 281 to enable fluid displacement cuff 246 to relax. As noted above, proceeding without deflating fluid displacement cuff 246 would make it difficult to infuse a medicinal agent in the desired treatment location defined by the proximal and distal flow restrictive cuffs. The logic then moves to decision block 311 to determine if the pressure timer has already been activated. An activated pressure timer indicates that the cuff inflation process (blocks 300c, 302, 303a and 309) was not an initial cuff inflation, but instead, was an inflation of the fluid displacement cuff during or near the end of an infusion cycle, as will become apparent below. If, in decision block 311, it is determined that the timer is already active, the logic moves to decision block 320, via connector H. The logical sequence after decision block 320 will be described in more detail below. If in decision block 311 it is determined that the timer is not already active, the timer is activated in block 304.

The logical sequence from block 304 to block 318 is identical to the logic described above relative to FIGS. 11A-11E. The next variation occurs after block 318, when bleed valve 281 is closed in block 319 (note block 318 no longer leads to block 320). After the bleed valve is closed, the logic returns to block 298a via connector G, and fluid displacement cuff 246 is once again placed in fluid communication with inflation pump 254. Note that when valve 266 is actuated, the pressure within proximal flow restrictive cuff 220, distal flow restrictive cuff 222 and flexible tubes 264 servicing the restrictive cuffs is not released, and thus, proximal flow restrictive cuff 220 and distal flow restrictive cuff 222 do not yet deflate.

The logic then advances through the cuff inflation sequence (blocks 300c, 302, 303a and 309) described above. At this time, the inflation pump is already activated, and the proximal and distal flow restriction cuffs are inflated. Thus, the inflation cycle first inflates fluid displacement cuff 246, and then deflates fluid the displacement cuff. After an infusion of a medicinal agent, this step will draw any residual medicinal agent back into the delivery catheter, thereby reducing the chance that any residual medicinal agent will migrate beyond the desired tissue location once the proximal and distal restriction cuffs are deflated. As noted above, if the timer is already on, the cuff inflation sequence will lead to decision block 320, via connector H.

At decision block 320, the logic loops until the inflation period is expired, and then the inflation pump is deactivated in block 322. At this time, valve 280 is still positioned such that fluid displacement cuff 246 is in fluid communication with the inflation pump, so that even with the inflation pump deactivated, the pressure within proximal flow restrictive cuff 220, distal flow restrictive cuff 222, and the flexible tubes 264 servicing the restrictive cuffs has not yet been released. Thus, in block 323a, valve 280 is moved to place proximal flow restrictive cuff 220 and distal flow restrictive cuff 222 in fluid communication with the deactivated inflation pump, thereby releasing the pressure and relaxing the proximal and distal flow restrictive cuffs. From that point on, the logical sequence for blocks 324-330 are identical to the sequences described above for FIG. 11A-11E. From block 330, the logical sequences all activate an inflation pump (block 300 for FIGS. 11A-C, block 297 for FIGS. 11D and 11E, and block 300c for FIG. 11F).

A separate figure for the logic employed to control automated system 250g (see FIG. 10G), which is an automated embodiment of the treatment process described in conjunction with FIG. 9, is not required, because the logic is virtually identical to the logic shown and described with respect to automated system 250a in FIG. 11A. The only difference is that automated system 250a simultaneously inflates (or deflates) a proximal and distal flow restrictive cuff at the same time, using a single inflation pump. In automated system 250g, the inflation pump inflates (or deflates) large flow restricting cuff 248, rather than the separate proximal and distal cuffs. Otherwise the logical sequence is unchanged.

Although the present invention has been described in connection with the preferred form of practicing it and modifications thereto, those of ordinary skill in the art will understand that many other modifications can be made to the present invention within the scope of the claims that follow. Accordingly, it is not intended that the scope of the invention in any way be limited by the above description, but instead be determined entirely by reference to the claims that follow.

The invention in which an exclusive right is claimed is defined by the following:

1. A method for delivering a medicinal agent to a treatment site within a limb of a patient, comprising the steps of:
(a) inserting a catheter into a blood vessel of the patient and advancing the catheter through the blood vessel until a distal tip of said catheter is disposed adjacent to the treatment site;
(b) stopping blood flow within the limb by applying an external pressure to the limb; and
(c) delivering the medicinal agent to the treatment site through the catheter so that the medicinal agent infuses the treatment site and remains at the treatment site at least while the blood flow in the limb is stopped.

2. The method of claim 1, further comprising the steps of:
(a) retaining the medicinal agent at the treatment site for a predetermined perfusion time to allow perfusion of the medicinal agent into tissue proximate the treatment site; and
(b) removing said external pressure to reestablish blood flow in the limb, after the predetermined perfusion time has elapsed.

3. The method of claim 2, wherein the perfusion time is less than about ten minutes.

4. The method of claim 2, further comprising the steps of:
(a) enabling blood flow to resume after the perfusion time, for a predetermined rest period, to reoxygenate tissue within the limb;
(b) again stopping the blood flow within the limb by reapplying the external pressure; and
(c) again delivering the medicinal agent to the treatment site through the catheter.

5. The method of claim 1, wherein the step of stopping blood flow within the limb comprises the step of applying an external pressure around the limb of the patient at a location proximal to the treatment site, said external pressure being sufficient to substantially interrupt blood flow through the limb past the point at which the pressure is applied.

6. The method of claim 5, wherein said external pressure is applied with a tourniquet, and the step of stopping blood flow within the limb further comprises the step of tightening said tourniquet around the limb of the patient at a point on the limb that is proximal to the treatment site.

7. The method of claim 5, wherein said external pressure is applied with a pressure cuff, and wherein the step of stopping blood flow within the limb comprises the step of inflating said pressure cuff around the limb of the patient at a point on the limb that is proximal to the treatment site.

8. The method of claim 5, further comprising the step of isolating said treatment site from a patient's tissue distal to said treatment site by applying an external pressure to the limb at a point on the limb that is distal to said treatment site.

9. The method of claim 8, wherein the step of isolating said treatment site comprises the step of inflating a pressure cuff around the limb of the patient at said point that is distal to the treatment site.

10. The method of claim 8, wherein the step of isolating said treatment site comprises the step of tightening a tourniquet around the limb of the patient at said point that distal to the treatment site.

11. The method of claim 1, further comprising the step of removing a substantial amount of fluid from the blood and lymph vessels adjacent to said treatment site by applying an external pressure to the limb in a region that substantially overlies said treatment site, after the step of inserting the catheter and before the step of stopping blood flow within the limb.

12. The method of claim 11, wherein the step of removing a substantial amount of fluid comprises the step of tightening a tourniquet around the limb of the patient at said region that substantially overlies said treatment site.

13. The method of claim 11, wherein the step of removing a substantial amount of fluid comprises the step of inflating a pressure cuff around the limb of the patient at said region that substantially overlies said treatment site.

14. The method of claim 1, further comprising the step of removing a substantial amount of fluid from the blood and lymph vessels adjacent to said treatment site by applying an external pressure to the limb at a region that substantially overlies said treatment site, after the step of inserting a catheter and before the step of stopping blood flow within the limb.

15. The method of claim 1, further comprising the step of displacing any residual medicinal agent back into the catheter from the treatment site by applying an external pressure to the limb at a region that substantially overlies said treatment site, after the step of delivering the medicinal agent to the treatment site.

16. The method of claim 15, wherein the step of displacing any residual medicinal agent comprises the step of tightening a tourniquet around the limb of the patient at said region that substantially overlies said treatment site.

17. The method of claim 15, wherein the step of displacing any residual medicinal agent comprises the step of inflating a pressure cuff around the limb of the patient at said region that substantially overlies said treatment site.

18. The method of claim 1, further comprising the step of applying an external pressure to the limb, over an area that overlies the treatment site and extends beyond said treatment site in both a proximal and a distal direction along the limb.

19. The method of claim 18, wherein the step of applying the external pressure to the limb over the area comprises the step of tightening a tourniquet around the limb of the patient.

20. The method of claim 18, wherein the step of applying the external pressure to the limb over the area comprises the step of inflating a pressure cuff around the limb of the patient.

21. The method of claim 1, wherein the step of inserting the catheter comprises the step of inserting the catheter into a vein of the patient and advancing the catheter in a retrograde direction within the vein until the distal end of the catheter is disposed adjacent to the treatment site.

22. The method of claim 21, wherein said catheter has a balloon disposed proximate the distal tip of said catheter, further comprising the step of isolating said treatment site from a patient's tissue distal to said treatment site by inflating the balloon of said catheter.

23. The method of claim 1, further comprising the step of administering to the treatment site a substance known to dilate and separate endothelial cells, thereby increasing a transfer of said medicinal agent across blood vessel walls at the treatment site.

24. The method of claim 23, wherein said substance comprises papaverine.

25. The method of claim 23, wherein said substance is utilized at least one of before, during, and after the step of delivering the medicinal agent to the treatment site.

26. A system for delivering and retaining a medicinal agent at a treatment site within a patient, comprising:
(a) an infusion catheter having a lumen that extends to a distal end of the infusion catheter from a port disposed adjacent to a proximate end of the catheter, said infusion catheter being adapted for insertion into a blood vessel of a patient and adapted to be advanced to a treatment site; and
(b) an external constrictor that is adapted to exert sufficient constrictive pressure on a limb of a patient to stop blood flow within the limb while a medicinal agent is infused into a treatment site to which the distal end of the catheter has been advanced through a blood vessel of a patient, so that the medicinal agent remains at a treatment site at least while a blood flow in a limb is stopped.

27. The system of claim 26, further comprising a delivery device connected in fluid communication with a proximal end of the lumen of the infusion catheter, said delivery device being employed to infuse the medicinal agent into the treatment site through the lumen.

28. The system of claim 27, wherein said delivery device comprises an infusion pump.

29. The system of claim 27, wherein said constrictor comprises a pressure actuated cuff, further comprising a controller connected to control operation of said delivery device and said pressure actuated cuff, wherein said controller automatically causes said pressure actuated cuff to stop a blood flow within a limb of a patient, and wherein said controller automatically activates said delivery device, causing the medicinal agent to be infused at the treatment site.

30. The system of claim 29, wherein said controller repetitively activates and deactivates said constrictor and said delivery device so that the medicinal agent is infused into the treatment site during successive cycles.

31. The system of claim 30, wherein said delivery device further comprises a flow sensor that produces a signal indicative of a flow of the medicinal fluid through the lumen, for use in determining a quantity of the medicinal agent delivered to the treatment site, said signal being supplied to the controller for use in controlling the delivery device.

32. The system of claim 29, wherein said controller comprises a timer that determines time intervals, including at least one of:
(a) a pressure time interval during which the controller causes a blood flow to be stopped in a limb of a patient;
(b) a rest time interval between successive cycles; and
(c) a dosage time interval during which the medicinal fluid is infused into the treatment site.

33. The system of claim 26, wherein said constrictor comprises a pressure actuated cuff adapted to wrap around a limb of a patient; further comprising an inflation pump operatively connected with said controller for receiving an activation signal from said controller, wherein said inflation pump is operatively connected to said cuff to provide a pressurized fluid for inflating said cuff.

34. The system of claim 33, wherein said pressure actuated cuff has a size and shape that enables the pressure actuated cuff to substantially overlap the treatment site, such that when said pressure actuated cuff is inflated, it is adapted to compress the treatment site, thereby forcing a substantial amount of fluid from the blood and lymph vessels within the treatment site.

35. The system of claim 33, wherein said constrictor further comprises a pressure sensor for detecting a pressure applied to the pressure actuated cuff, producing a pressure signal that is indicative of the pressure of the fluid applied to inflate the pressure actuated cuff.

36. The system of claim 29, wherein said controller determines a total dosage of the medicinal agent that is delivered to the treatment site.

37. The system of claim 36, wherein said controller causes the delivery device to deliver a predetermined dosage of the medicinal agent.

38. The system of claim 26, wherein said infusion catheter includes a radio-opaque element disposed adjacent to its distal end to assist in positioning the distal end adjacent to the treatment site.

39. The system of claim 26, wherein said infusion catheter includes an enlarged distal portion adjacent to the distal end of the infusion catheter adapted for sealing against an inner wall of a blood vessel to prevent the medicinal agent from flowing back along the infusion catheter and away from the treatment site.

40. The system of claim 26, wherein said infusion catheter includes a second lumen adapted to receive a guide wire to assist in positioning the distal end of the infusion catheter.

41. The system of claim 26, further comprising an introducer sheath, adapted for insertion into a blood vessel of a patient, to provide a reusable access site for insertion of the infusion catheter.

42. The system of claim 29, further comprising an additional external constrictor that is adapted to exert sufficient constrictive pressure on a limb of a patient at a location distal to said treatment site to prevent a medicinal agent infused into a treatment site from migrating away from said treatment site and into tissue located distal to said treatment site.

43. The system of claim 42, wherein said additional external constrictor comprises another pressure actuated cuff adapted to wrap around a limb of a patient; further comprising an inflation pump operatively connected to said controller for receiving an activation signal from said controller, and wherein said inflation pump is operatively connected to the other pressure actuated cuff to provide a pressurized fluid for inflating said other pressure actuated cuff.

44. The system of claim 43, wherein the pressure actuated cuffs comprising said constrictor and said additional external constrictor are both inflated by the inflation pump.

45. The system of claim 44, further comprising a valve in fluid communication with said inflation pump and the pressure actuated cuffs comprising said constrictor and said additional external constrictor, said valve being controllably connected to said controller, such that upon receiving an activation signal from said controller, said valve selectively enables one of the pressure actuated cuffs comprising said constrictor and said additional external constrictor to be inflated by said inflation pump.

46. The system of claim 43, further comprising another inflation pump, wherein the pressure actuated cuffs comprising said constrictor and said additional external constrictor are each inflated by a different inflation pump.

47. The system of claim 43, further comprising a fluid displacement cuff having a size and shape sufficient to enable the fluid displacement cuff to substantially overlie the treatment site, such that when said fluid displacement cuff is activated, it is adapted to compress the treatment site, thereby displacing a substantial amount of fluid from blood and lymph vessels proximate to the treatment site.

48. The system of claim 47, further comprising an inflation pump operatively connected to said controller for receiving an activation signal from said controller, wherein said inflation pump is operatively connected to said fluid displacement cuff to provide pressurized fluid for activating the fluid displacement cuff by inflating said fluid displacement cuff with the pressurized fluid.

49. The system of claim 47, further comprising a plurality of inflation pumps, wherein the pressure actuated cuff comprising said constrictor, the pressure actuated cuff comprising said additional external constrictor, and the fluid displacement cuff are each inflated by a different inflation pump.

50. The system of claim 47, further comprising another inflation pump, wherein the actuated cuff comprising said constrictor and the actuated cuff comprising said additional external constrictor are inflated by the same inflation pump, and the fluid displacement cuff is inflated by a different inflation pump.

51. The system of claim 47, wherein the pressure actuated cuff comprising said constrictor, the pressure actuated cuff comprising said additional external constrictor, and the fluid displacement cuff are each inflated by the inflation pump.

52. The system of claim 51, further comprising a valve in fluid communication with said inflation pump and the pressure actuated cuff comprising said constrictor, the pressure actuated cuff comprising said additional external constrictor, and the fluid displacement cuff, said valve being controllably connected to said controller such that upon receiving an activation signal from said controller, said valve selectively enables one of the pressure actuated cuffs and the fluid displacement cuff to be inflated by said inflation pump.

53. The system of claim 52, further comprising a bleed valve in fluid communication with said fluid displacement cuff, said bleed valve being controllably connected to said controller such that upon receiving an activation signal from said controller, said bleed valve enables the fluid displacement cuff to be deflated without also deflating either pressure actuated cuff.

54. The system of claim 26, wherein said infusion catheter includes an inflatable balloon disposed adjacent to the distal end of the infusion catheter and adapted for sealing against an inner wall of a blood vessel to prevent the medicinal agent from flowing back along the infusion catheter and away from the treatment site, and further comprising a balloon inflation pump operatively connected to said controller for receiving an activation signal from said controller, said balloon inflation pump being operatively connected to said infusion catheter to provide a pressurized fluid for inflating said balloon.

55. A method for controlling delivery of a medicinal agent to a treatment site within a limb of a patient through a catheter that has been inserted into a blood vessel of the patient and advanced to the treatment site, comprising the steps of:
(a) automatically activating a constrictor, causing the constrictor to apply an external pressure to the limb of the patient to stop the flow of blood within the limb; and
(b) automatically activating a delivery device to deliver the medicinal agent to the treatment site through the catheter, said medicinal agent remaining at the treatment site at least while the flow of blood within the limb is stopped.

56. The method of claim 55, further comprising the step of releasing the external pressure applied by the constrictor after a predetermined constriction period of time has elapsed.

57. The method of claim 55, wherein the step of automatically activating the delivery device comprises the step of infusing the medicinal agent for a predetermined infusion period of time.

58. The method of claim 55, further comprising the step of delivering a predetermined dosage of the medicinal agent to the treatment site.

59. The method of claim 55, further comprising the steps of:
(a) detecting a quantity of the medicinal agent delivered to the treatment site; and
(b) determining when to deactivate said delivery device as a function of the quantity of the medicinal agent that has been delivered to the treatment site.

60. The method of claim 55, further comprising the step of deactivating said delivery device once a total desired quantity of the medicinal agent delivered to the treatment site equals a predetermined threshold limit.

61. The method of claim 55, further comprising the steps of:
(a) repeating steps (a) and (b) in a plurality of successive cycles; and
(b) allowing blood flow to resume in the limb for a predetermined rest period between successive cycles.

62. The method of claim 55, further comprising the step of automatically activating a second constrictor disposed at a point on the limb that is distal to treatment site, causing the second constrictor to apply an external pressure to the limb of the patient sufficient to prevent any medicinal agent delivered to said treatment site from migrating to tissue distal to the treatment site, such that said second constrictor is activated whenever the constrictor is activated.

63. The method of claim 55, further comprising the step of automatically activating a fluid displacement cuff that substantially overlies said treatment site before activating the constrictor, causing a substantial volume of fluid to be displaced from the treatment site.

64. The method of claim 63, further comprising the step of automatically activating said fluid displacement cuff after activating a delivery device, causing any residual medicinal agent delivered to the treatment site to be displaced back into said delivery device.

65. A machine readable medium on which are stored machine readable instructions for performing the steps of claim 55.
